SEQUENCE LISTING (4 sequences)

SEQ ID NO: 1 - DNA, 20 bp, synthetic construct
  source 1..20: mol_type = other DNA
  modified_base 1: mod_base = OTHER; note = 5' Phosphate
aatgatacggcgaccaccga

SEQ ID NO: 2 - DNA, 24 bp, synthetic construct
  source 1..24: mol_type = other DNA
caagcagaagacggcatacgagat

SEQ ID NO: 3 - DNA, 44 bp, synthetic construct
  source 1..44: mol_type = other DNA
tcggtggtcgccgtatcattcaagcagaagacggcatacgagat

SEQ ID NO: 4 - DNA, 24 bp, synthetic construct
  source 1..24: mol_type = other DNA
atctcgtatgccgtcttctgcttg

(19) United States
(12) Patent Application Publication
(10) Pub. No.: US 2024/0318247 A1
(43) Pub. Date: Sep. 26, 2024

(54) COMPOSITIONS AND METHODS FOR DENSLEY-PACKED ANALYTE ANALYSIS

(71) Applicant: Pacific Biosciences of California, Inc., Menlo Park, CA (US)

(72) Inventors: Bojan BERGHUIS, Pleasanton, CA (US); Bryan STAKER, San Ramon, CA (US)

(21) Appl. No.: 18/587,683

(22) Filed: Feb. 26, 2024

Related U.S. Application Data
(63) Continuation of application No. PCT/US2022/041529, filed on Aug. 25, 2022.
(60) Provisional application No. 63/238,722, filed on Aug. 30, 2021.
(60) Provisional application No. 63/238,087, filed on Aug. 27, 2021.

(51) Int. Cl. C12Q 1/6874; C12Q 1/689

(52) U.S. Cl. CPC C12Q 1/6874; C12Q 1/689

(57) ABSTRACT

Disclosed herein are methods and systems for detection and discrimination of optical signals from a densely packed substrate. These have broad applications for biomolecule detection near or below the diffraction limit of optical systems, including in improving the efficiency and accuracy of polynucleotide sequencing applications.

### CROSS-REFERENCE

This application is a continuation of International Application No. PCT/US2022/041529, filed Aug. 25, 2022, which claims the benefit of U.S. Provisional Application No. 63/238,087, filed Aug. 27, 2021, and U.S. Provisional Application No. 63/238,722, filed Aug. 30, 2021, which are herein incorporated by reference in their entireties.

The instant application contains a Sequence Listing which has been submitted in ASCII format via EFS-Web and hereby incorporated by reference in its entirety. Said ASCII copy, created on Oct. 13, 2022, is named 55495-814-301_SL.xml and is 5,177 bytes in size.

### BACKGROUND

Affordable, rapid sequencing is causing a revolution in medicine and healthcare globally. As price of a genome has dropped dramatically since the first human genome was sequenced in year 2000, a significant milestone, $1,000 genome, was recently achieved. However, there is huge demand for lower cost sequencing that can enable applications such as large population sequencing, disease screening and early detection.

A standard for measuring the cost of sequencing is the price of a 30× human genome, defined as 90 gigabases. The major cost components for sequencing systems are primarily the consumables which include biochip and reagents and secondarily the instrument costs.

### SUMMARY

In certain aspects, described herein is a system comprising an analyte disposed adjacent to a substrate, wherein said analyte has a first dimension and a second dimension, wherein said first dimension is along an axis parallel to said substrate and said second dimension is along an axis orthogonal to said substrate, wherein said first dimension is less than a diffraction limit of an optical system (λ/(2*NA)) configured to image said analyte, and wherein said second dimension is less than one-half of a depth-of-focus of said optical system (λ/(2*NA\hat{ }2)). In some embodiments, said analyte is a nucleic acid concatemer. In some embodiments, said analyte is a protein. In some embodiments, said analyte is deoxyribonucleic acid (DNA), ribonucleic acid (RNA), messenger ribonucleic acid (mRNA), or any combination thereof. In some embodiments, said DNA or RNA is single stranded. In some embodiments, said one or more analytes are bound to a support, wherein said support is immobilized on said substrate. In some embodiments, said support is UV treated. In some embodiments, said support is spherical or circular. In some embodiments, said support is a nucleic acid origami structure. In some embodiments, said nucleic acid origami structure comprises a nucleic acid molecule. In some embodiments, said nucleic acid molecule is DNA or RNA. In some embodiments, said DNA or RNA is single stranded. In some embodiments, said support is a circular disk and said analyte is bound to a single side of said circular disk. In some embodiments, said support is a metal or non-metal nanoball. In some embodiments, said metal or non-metal nanoball comprises carbon. In some embodiments, said support comprises linkers configured to bind to said analyte. In some embodiments, said linkers are nucleic acid primers. In some embodiments, said analyte comprises repeating regions. In some embodiments, said linkers are configured to bind to said repeating regions of said analyte. In some embodiments, said linkers comprise nucleic acid molecules. In some embodiments, said nucleic acid molecules are DNA or RNA. In some embodiments, said DNA or RNA is double stranded. In some embodiments, said DNA or RNA is single stranded. In some embodiments, said repeating regions comprise one or more known sequences. In some embodiments, said one or more known sequences comprise a barcode sequence, an adaptor sequence, a primer sequence, or any combination thereof. In some embodiments, a melting point of said linkers is greater than a temperature reached during processing of said analyte. In some embodiments, said substrate comprises one or more artifacts adjacent to said analyte, wherein said one or more artifacts do not generate a signal. In some embodiments, said one or more artifacts generate a neighboring effect on said analyte. In some embodiments, said neighboring effect comprises immobilizing analyte. In some embodiments, at least 10% of said one or more artifacts comprise a first dimension along the axis parallel to the substrate of less than the diffraction limit of said optical system configured to image said analyte when said one or more artifacts are deposited adjacent to said analyte, and wherein at least 10% of said one or more artifacts comprise a second dimension along the axis orthogonal to the substrate of less than one-half of the depth-of-focus of said optical system configured to image said analyte when said one or more artifacts are deposited adjacent to said analyte. In some embodiments, said analyte comprises a scaffold. In some embodiments, said scaffold comprises at least two scaffolds wherein said at least two scaffolds are double stranded and intersect at a point of intersection. In some embodiments, said point of intersection is a Holliday junction. In some embodiments, at said point of intersection said at least two scaffolds are bound together. In some embodiments, said scaffold comprises one or more biopolymers. In some embodiments, said one or more biopolymers comprise a carbon-based polymer. In some embodiments, said one or more biopolymers comprise a polyether. In some embodiments, said one or more biopolymers comprise a polypeptide. In some embodiments, said one or more biopolymers are detergent molecules. In some embodiments, said one or more biopolymers comprise a nucleic acid molecule. In some embodiments, said nucleic acid molecule is DNA or RNA. In some embodiments, said one or more biopolymers is single stranded DNA, and said scaffold comprises at least two of said one or more biopolymers. In some embodiments, said two biopolymers are oriented in a same direction. In some embodiments, said same direction is 5′ to 3′. In some embodiments, the 5′ terminal bases of said two biopolymers are linked via a linker. In some embodiments, said linker is a covalent linker. In some embodiments, said linker is an additional biopolymer. In some embodiments, said additional biopolymer is a polypeptide. In some embodiments, said additional biopolymer is an additional nucleic acid molecule. In some embodiments, said nucleic acid molecule is DNA. In some embodiments, said biopolymer comprises 1 to 500 monomers. In some embodiments, said biopolymer is double stranded DNA and comprises 100 base pairs. In some embodiments, said scaffold is configured to bind to repeating regions of said analyte. In some embodiments, said repeating regions comprise one or more known sequences. In some embodiments, said one or more known sequences comprise a barcode sequence, an adaptor sequence, a primer sequence, or any combination thereof. In some embodiments, a melting point of said scaffold is greater than a temperature reached during processing of said analytes.

In certain aspects, described herein is a method for processing one or more analytes, the method comprising: (a) depositing said one or more analytes adjacent to a substrate, wherein at least 10% of said one or more analytes comprise a first dimension along the axis parallel to the substrate of less than the diffraction limit of an optical system configured to image said one or more analytes when said one or more analytes are deposited adjacent to said substrate, and wherein at least 10% of said one or more analytes comprise a second dimension along the axis orthogonal to the substrate of less than one-half of the depth-of-focus of said optical system configured to image said one or more analytes when said one or more analytes are deposited adjacent to said substrate. (b) contacting said one or more analytes with a plurality of probes over a plurality of cycles, wherein said plurality of probes generate a plurality of signals; (c) obtaining said plurality of optical signals from said plurality of probes over said plurality of cycles of said plurality of probes binding to said one or more analytes deposited adjacent to said substrate; and (d) processing at least one optical signal of said plurality of optical signals to identify said one or more analytes of said plurality of analytes. In some embodiments, said one or more analytes are nucleic acid concatemers. In some embodiments, said one or more analytes are proteins. In some embodiments, said one or more analytes are DNA, RNA, mRNA, or any combination thereof. In some embodiments, said DNA or RNA is single stranded. In some embodiments, said one or more analytes are bound to one or more support structures, wherein said one or more support structures are immobilized on said substrate. In some embodiments, said one or more support structures are UV treated. In some embodiments, a single analyte of said one or more analytes is bound to a single support structure of said one or more support structures. In some embodiments, a single analyte of said one or more analytes is bound to a plurality of support structures of said one or more support structures. In some embodiments, said support structure is spherical or circular. In some embodiments, said support structure is a nucleic acid origami structure. In some embodiments, said nucleic acid origami structure comprises a nucleic acid molecule. In some embodiments, said nucleic acid molecule is DNA or RNA. In some embodiments, said DNA or RNA is single stranded. In some embodiments, said support structure is a circular disk and the one or more analytes are bound to a single side of the circular disk. In some embodiments, said support structure is a metal or non-metal nanoball. In some embodiments, said metal or non-metal nanoball comprises carbon. In some embodiments, said support structure comprises linkers configured to bind to said one or more analytes. In some embodiments, said linkers are nucleic acid primers. In some embodiments, said one or more analytes comprise repeating regions. In some embodiments, said linkers are configured to bind to said repeating regions of said one or more analytes. In some embodiments, said linkers comprise nucleic acid molecules. In some embodiments, said nucleic acid molecules are DNA or RNA. In some embodiments, said support structure comprises one or more biopolymers. In some embodiments, said one or more biopolymers is single stranded DNA, and said support structure comprises at least two of said one or more biopolymers. In some embodiments, said two biopolymers are oriented in a same direction. In some embodiments, said same direction is 5′ to 3′. In some embodiments, the 5′ terminal bases of said two biopolymers are linked via a linker. In some embodiments, said linker is a covalent linker. In some embodiments, said linker is an additional biopolymer In some embodiments, said additional biopolymer is a polypeptide. In some embodiments, said additional biopolymer is an additional nucleic acid molecule. In some embodiments, said repeating regions comprise one or more known sequences. In some embodiments, said one or more known sequences comprise a barcode sequence, an adaptor sequence, a primer sequence, or any combination thereof. In some embodiments, a melting point of said linkers is greater than a temperature reached during processing of said one or more analytes. In some embodiments, said substrate comprises one or more artifacts adjacent to said one or more analytes, wherein said one or more artifacts do not generate a signal. In some embodiments, said one or more artifacts generate a neighboring effect on said one or more analytes. In some embodiments, said neighboring effect comprises immobilizing said one or more analytes. In some embodiments, said one or more analytes comprise a scaffold. In some embodiments, said scaffold comprises at least two scaffolds wherein said at least two scaffolds are double stranded and intersect at a point of intersection. In some embodiments, said scaffold comprises one or more biopolymers. In some embodiments, said one or more biopolymers comprise a carbon-based polymer, a polypeptide, a detergent molecule, or a nucleic acid molecule. In some embodiments, said nucleic acid molecule is DNA or RNA. In some embodiments, said DNA or RNA is double stranded. In some embodiments, said DNA or RNA is single stranded. In some embodiments, said biopolymer comprises 1 to 500 monomers. In some embodiments, said biopolymer is double stranded DNA and comprises 100 base pairs. In some embodiments, said scaffold is configured to bind to repeating regions of said one or more analytes. In some embodiments, said repeating regions comprise a one or more known sequences. In some embodiments, said one or more known sequences comprise a barcode sequence, an adaptor sequence, a primer sequence, or any combination thereof. In some embodiments, a melting point of said scaffold is greater than a temperature reached during processing of said one or more analytes. In some embodiments, a surface of said substrate comprises one or more anchor moieties configured to bind to said one or more analytes. In some embodiments, said one or more anchor moieties comprise an antibody. In some embodiments, said one or more anchor moieties are nucleic acid primers. In some embodiments, said one or more anchor moieties are configured to bind to repeating regions of said one or more analytes. In some embodiments, said one or more anchor moieties comprise nucleic acid molecules. In some embodiments, said repeating regions comprise a one or more known sequences. In some embodiments, said one or more known sequences comprise a barcode sequence, an adaptor sequence, a primer sequence, or any combination thereof. In some embodiments, said surface comprises one or more reagents to immobilize to one or more anchor moieties. In some embodiments, said one or more reagents comprises streptavidin, polyethylene glycol (PEG), biotin, or any combination thereof. In some embodiments, a melting point of said one or more anchor moieties is greater than a temperature reached during processing of said one or more analytes. In some embodiments, a density of said one or more analytes does not exceed about 25 analytes per square micrometer. In some embodiments, said one or more analytes first dimension along the axis parallel to the substrate are less than 400 nm.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a comparison of measured full-width half maximum widths (FWHM) for DNA circularly amplified concatemers (CATs) of 5 min, 10 min, 15 min & 60 min CATs of approximately 12 kB, 24 kB, 36 kB and 144 kB of DNA nucleotide length.

FIG. 2 shows contours of constant CAT first dimension along the axis parallel to the substrate, a comparison of CAT diameter with replication time.

FIG. 3 illustrates a self-assembled surface with a mixture of both sequencing (light) CATs and dark CATs.

FIG. 4 compares compactification density against CAT diameter for various sizes of CATs.

FIG. 5 depicts an example of a computer system for use in the methods described herein.

FIG. 6A illustrates a DNA origami support structure containing primers to which CATs bind.

FIG. 6B shows DNA origami forming a block/disk structure, one side of which binds to a surface, the other side of which binds primers capable of binding CATs.

FIG. 6C illustrates DNA origami structures covalently crosslinked to become resistant to sequencing related temperature cycling.

FIG. 7A illustrates a functionalized carbon nanoball with primers which bind CATs providing 3-dimensional structural support.

FIG. 7B shows an example illustrating the CAT-carbon nanoball loading process.

FIG. 8 shows a single CAT that has multiple substrates, including possibly origami, carbon nanoball or proteins functionalized with linkers

FIG. 9A shows double stranded DNA (dsDNA) giving 3-dimensional support to single stranded DNA CATs.

FIG. 9B illustrates that dsDNA scaffolding can also come in the form of Holliday junctions.

FIG. 10 illustrates how dsDNA can add more structure to a CAT in comparison to ssDNA.

FIG. 11 shows staple primers being supplied to a CAT which results in compactifying and stabilizing the ssDNA.

FIG. 12 illustrates a surface loaded with sequencing CATs and dark CATs.

FIG. 13 shows a diagram of binding strength versus specificity space (black), listing examples (non-exhaustive) of types of binding (blue) that fall within a quadrant.

FIG. 14 illustrates CAT binding to PEG surface with dsDNA handles.

FIG. 15 shows examples of crosslinker molecules that do not rely on nucleic acid hybridization, but covalent bonding between the DNA primers and a polymer or biomolecule such as (but not limited to) a peptide, sugar molecule, PEG or detergent which can also have additional functionality, for example a pH change that switches a peptide from a relaxed to a condensed state (e.g. alpha helix).

FIG. 16 shows that using the molecules described in FIG. 14, a CAT can be condensed by inducing the conformation change of the crosslinker molecules.

FIG. 17 shows that non-nucleic acid, biopolymer based crosslinkers can also contain a reactive group (e.g. a cysteine amino acid, that reacts specifically with only other free cysteines to form a covalent bond) that links two crosslinkers together

FIG. 18 illustrates how the idea in FIG. 16 can be expanded to multiple linkers, which might be a useful way to link a multitude of crosslinkers together.

FIG. 19A shows a drawing which illustrates the wrapping of a long strand of ssDNA around a nanorod solid structure, during CAT creation

FIG. 19B shows a drawing which illustrates the hybridization of a CAT onto multiple primers attached on a solid support surface (nanorod) which provides an anchoring point for the CAT molecule to condense into and the hybridization to multiple primers ensures secure attachment.

FIG. 20A shows a drawing representing a set of two or more “non-hairpin” primers (Primer A and Primer B) in which Primer A contains a 5′ tail and Primer B contains the complement of the 5′ tail of Primer 1. The 5′ region hybridizes with the 5′ tail of the complement primer and provides structural rigidity to reduce physical size of the loaded DNA sample. The 3′ prime end can hybridize to one or more locations on the sequencing library adaptor region including: forward sequencing primer, reverse sequencing primer, any barcode primer regions, or any combination of the previous.

FIG. 20B shows a drawing of a combinations of primers capable of hybridizing to any region the library adaptor region and contains either 5′ palindromic (hairpin) or 5′ non-hairpin primers or any combination of hairpin and non-hairpin 5′ regions.

FIG. 20C shows a drawing illustrating that the 5′ Hairpin or non-hairpin region can be universal, in which any 3′ primer has a 5′ hairpin or non-hairpin that can hybridize to the 5′ hairpin or non-hairpin of any other primer regardless of location on the library adaptor or the 5′ hairpin or 5′ non-hairpin region can be unique, in which the 3′ primer can only hybridize to a second primer which hybridizes to the same location on the library adaptor

FIG. 21 shows molecule on a surface without nanoarray passivation (right), compared to molecules on a surface with nanoarray passivation (left, purple circles) where d is spacing between spots, s is the binding site size.

FIG. 22 shows a schematic illustration of the molecule patterning process through 2D nanosphere close-packing, selective passivation, lift-off, and finally, DNA molecules placement.

FIG. 23 shows a table demonstrating nanosphere diameter-dependent density.

FIG. 24 shows G4 motifs as a quartet (left) which are thought to provide functional secondary and tertiary structure via the formation of G4 DNA (also known as G-quadruplexes; right).

FIG. 25 shows an example modified Y-shaped adaptor, the ligation of which would allow for introduction of a G4 motif.

FIG. 26 depicts CATs binding to a streptavidin-functionalized surface.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems and methods that facilitate optical detection and discrimination of probes bound to tightly packed analytes bound to the surface of a substrate. In part, the methods and systems described herein rely on repeated detection of a plurality of target analytes on the surface of a substrate to improve the accuracy of identification of a relative location of each analyte on the substrate. This information can then be used to perform signal resolving on each image of a field of the substrate for each cycle to reliably identify a signal from a probe bound to the target analyte. In some embodiments, the resolving comprises deconvolution. In some embodiments, this type of deconvolution processing can be used to distinguish between different probes bound to the target analyte that have overlapping emission spectrum when activated by an activating light. In some embodiments, the deconvolution processing can be used to separate optical signals from neighboring analytes. This is especially useful for substrates with analytes having a density wherein optical detection is challenging due to the diffraction limit of optical systems.

In some embodiments, the methods and systems described herein are particularly useful in sequencing. By providing methods and systems that facilitate reliable optical detection on densely packed substrates, costs associated with sequencing, such as reagents, number of clonal molecules used, processing and read time, can all be reduced to greatly advance sequencing technologies, specifically, sequencing by synthesis using optically detected nucleotides.

Although the systems and methods described herein have important implications for advancing sequencing technology, the methods and systems described herein are generally applicable to optical detection of analytes bound to the surface of a substrate, including on the single molecule level.

The systems and method for imaging analytes disposed on a substrate with a sub-diffraction limited optical system are described in U.S. Ser. No. 11/047,005 and PCT/US2019/051796 the contents of which are incorporated by reference herein

### Sequencing Cost Reduction

Sequencing technologies include image-based systems developed by companies such as Illumina and Complete Genomics and electrical based systems developed by companies such as Ion Torrent and Oxford Nanopore. Image-based sequencing systems currently have the lowest sequencing costs of all existing sequencing technologies. Image-based systems achieve low cost through the combination of high throughput imaging optics and low-cost consumables. However, prior art optical detection systems have minimum center-to-center spacing between adjacent resolvable molecules of about a micron, in part due to the diffraction limit of optical systems. In some embodiments, described herein are methods for attaining significantly lower costs for an image-based sequencing system using existing biochemistries using cycled detection, determination of precise positions of analytes, and use of the positional information for highly accurate deconvolution of imaged signals to accommodate increased packing densities below the diffraction limit.

### High Density Distributions of Analytes on a Surface of a Substrate

In some embodiments there exists a high-density region of 80 nm diameter binding regions (spots) on a 240 nm pitch. In this embodiment, an ordered array can be used where single-stranded DNA molecule exclusively binds to specified regions on chip. In some embodiments, concatemers (i.e., a long continuous DNA molecule that contains multiple copies of the same DNA sequence linked in series) smaller than 40 kB are used so as to not overfill the spot. The size of the concatemers scales roughly with area, meaning the projected length of the smaller concatemer may be approximate 4 kB to 5 kB resulting in approximately 10 copies if the same amplification process is used. It is also possible to use 4 kB lengths of DNA and sequence each concatemer directly. Another option is to bind a shorter segment of DNA with unsequenced filler DNA to bring the total length up to the size needed to create an exclusionary molecule.

In a comparison of the proposed pitch compared to a sample effective pitch used for a S1,000 genome, the density of the new array is 170-fold higher, meeting the criteria of achieving 100-fold higher density. The number of copies per imaging spot per unit area also meets the criteria of being at least 100-fold lower than the prior existing platform. This helps ensure that the reagent costs are 100-fold more cost effective than baseline.

### Imaging Densely Packed Single Biomolecules and the Diffraction Limit

One constraint for increased molecular density for an imaging platform is the diffraction limit. The equation for the diffraction limit of an optical system is:

$$D = λ / 2 NA$$

where D is the diffraction limit, λ, is the wavelength of light, and NA is the numerical aperture of the optical system. Typical air imaging systems have NA's of 1.0 to 1.2. Using λ, =600 nm, the diffraction limit is between 250 nm and 300 nm. For a water immersion system, the NA is −1.0, giving a diffraction limit of 300 nm.

If features on an array or other substrate surface comprising biomolecules are too close, two optical signals may overlap substantially so that you just see a single blob that cannot be reliably resolved based on the image alone. This can be exacerbated by errors introduced by the optical imaging system, such as blur due to inaccurate tracking of a moving substrate, or optical variations in the light path between the sensor and the surface of a substrate.

The transmitted light or fluorescence emission wavefronts emanating from a point in the specimen plane of the microscope become diffracted at the edges of the objective aperture, effectively spreading the wavefronts to produce an image of the point source that is broadened into a diffraction pattern having a central disk of finite, but larger size than the original point. Therefore, due to diffraction of light, the image of a specimen never perfectly represents the real details present in the specimen because there is a lower limit below which the microscope optical system cannot resolve structural details.

The observation of sub-wavelength structures with microscopes is difficult because of the diffraction limit. A point object in a microscope, such as a fluorescent protein or polynucleotide, may generate an image at the intermediate plane that may include a diffraction pattern created by the action of interference. When highly magnified, the diffraction pattern of the point object may be observed to include a central spot (diffraction disk) surrounded by a series of diffraction rings. Combined, this point source diffraction pattern is referred to as an Airy disk.

The size of the central spot in the Airy pattern is related to the wavelength of light and the aperture angle of the objective. For a microscope objective, the aperture angle is described by the numerical aperture (NA), which includes the term sin (θ), the half angle over which the objective can gather light from the specimen. In terms of resolution, the radius of the diffraction Airy disk in the lateral (x,y) image plane is defined by the following formula: Abbe Resolution=λ/2*NA, where λ is the average wavelength of illumination in transmitted light or the excitation wavelength band in fluorescence. The objective numerical aperture (NA=n·sin(θ)) is defined by the refractive index of the imaging medium (n; usually air, water, glycerin, or oil) multiplied by the sine of the aperture angle (sin(θ)). As a result of this relationship, the size of the spot created by a point source decreases with decreasing wavelength and increasing numerical aperture, but always remains a disk of finite diameter. The Abbe resolution (i.e., Abbe limit) is also referred to herein as the diffraction limit and defines the resolution limit of the optical system.

If the distance between the two Airy disks or point-spread functions is greater than the diffraction limit, the two point sources are considered to be resolved (and can readily be distinguished). Otherwise, the Airy disks merge together and are considered not to be resolved.

Thus, light emitted from a detectable label point source with wavelength 2, traveling in a medium with refractive index n and converging to a spot with half-angle 0 may make a diffraction limited spot with a diameter: d=λ/2*NA. Considering green light around 500 nm and a NA (Numerical Aperture) of 1, the diffraction limit is roughly d=λ/2=250 nm (0.25 pm), which limits the density of analytes such as proteins, nucleotides and other sequencing substrates on a surface able to be imaged by conventional imaging techniques. As used herein, sequencing substrates include any analyte that sequence information can be derived from, such as a template for a sequencing reaction. Even in cases where an optical microscope is equipped with the highest available quality of lens elements, is perfectly aligned, and has the highest numerical aperture, the resolution remains limited to approximately half the wavelength of light in the best-case scenario. To increase the resolution, shorter wavelengths can be used such as UV and X-ray microscopes. These techniques offer better resolution but are expensive, suffer from lack of contrast in biological samples and may damage the sample.

### Densely-Packed Analyte Layers and Detection Methods

Provided herein are systems and methods to facilitate imaging of signals from analytes deposited on a surface with a center-to-center spacing below the diffraction limit. These systems and methods use advanced imaging systems to generate super-resolution images, and cycled detection to facilitate positional determination of molecules on the substrate with high accuracy and resolving of images to obtain signal identity for each molecule on a densely packed surface with high accuracy. These methods and systems allow sequencing by synthesis on a densely packed substrate to provide highly efficient and very high throughput polynucleotide sequence determination with high accuracy.

The major cost components for sequencing systems are primarily the consumables which include biochip and reagents and secondarily the instrument costs. To reach a S10 30× genome, a 100-fold cost reduction, the amount of data per unit area needs to increase by 100-fold and the amount of reagent per data point needs to drop by 100-fold.

### Image Resolving

In some embodiments, the image resolving methods described herein comprise deconvolution. Deconvolution is an algorithm-based process used to reverse the effects of convolution on recorded data. The concept of deconvolution is widely used in the techniques of signal processing and image processing. Because these techniques are in turn widely used in many scientific and engineering disciplines, deconvolution finds many applications.

In optics and imaging, the term “deconvolution” is specifically used to refer to the process of reversing the optical distortion that takes place in an optical microscope, electron microscope, telescope, or other imaging instrument, thus creating clearer images. It is usually done in the digital domain by a software algorithm, as part of a suite of microscope image processing techniques.

The usual method is to assume that the optical path through the instrument is optically perfect, convolved with a point spread function (PSF), that is, a mathematical function that describes the distortion in terms of the pathway a theoretical point source of light (or other waves) takes through the instrument. Usually, such a point source contributes a small area of fuzziness to the final image. If this function can be determined, it is then a matter of computing its inverse or complementary function, and convolving the acquired image with that. Deconvolution maps to division in the Fourier co-domain. This allows deconvolution to be easily applied with experimental data that are subject to a Fourier transform. An example is NMR spectroscopy where the data are recorded in the time domain, but analyzed in the frequency domain. Division of the time-domain data by an exponential function has the effect of reducing the width of Lorenzian lines in the frequency domain. The result is the original, undistorted image.

However, for diffraction limited imaging, deconvolution is also needed to further refine the signals to improve resolution beyond the diffraction limit, even if the point spread function is perfectly known. It is very hard to separate two objects reliably at distances smaller than the Nyquist distance. However, described herein are methods and systems using cycled detection, analyte position determination, alignment, and deconvolution to reliably detect objects separated by distances much smaller than the Nyquist distance.

### Making High Density Random Layers of Concatemers for Sequencing

Also provided herein are methods of making and using high density concatemer layers. In some embodiments, the concatemers are randomly distributed on a surface of a substrate in a close-packed layer for individual detection and sequencing. In some embodiments, provided herein are methods of making and randomly distributing a layer of concatemers on a substrate such that they achieve a high density or average center-to-center distance.

Concatemers (i.e., CATs), are long single-stranded DNA molecules made through rolling circle amplification (RCA) of a ssCircular DNA. In some embodiments, the concatemers each comprise from a few up to several hundred copies of a target DNA sequence inserted between known sequence adapters. A library of concatemers comprising target DNA sequences can be generated. In some embodiments, the concatemers comprise features that self-exclude to facilitate layering a close-packed single layer of concatemers on a substrate with minimal overlap or a minimum distance between adjacent concatemers and without needing specific attachment points on the substrate. These exclusionary features facilitate close-packed layers while minimizing the number of nearest neighbor concatemers that are too close to be resolved by optical imaging, as described herein.

In some embodiments, provided herein are substrates comprising a surface, wherein the surface is bound to a close-packed, randomly distributed collection of amplified targets, such as DNA concatemers.

In some embodiments, this substrate is used to facilitate nucleotide sequencing, including of whole genomes or exomes. In some embodiments, large numbers of individual cellular targets can be sequenced. These can represent a selected panel of targets using cluster sequencing. Sequencing as described herein can be used, for example, to (i) detect multiple genetic variants (e.g., for genotyping, drug resistance determination, paternity, or identification), (ii) sequence multiple cDNA molecules for gene expression analysis for enumeration of pathway dynamics, or (iii) detect methylated residues on a target polynucleotide following bi-sulphite treatment. In some embodiments, sequencing methods require target amplification to generate small clusters of—200 target copies as described in the embodiments.

The method, in one embodiment, comprises: the creation of circularized single stranded molecules for targets across the genome using ligase reactions, amplification of the circularized DNA using isothermal whole genome amplification methods to generate clusters of circularized amplified targets (CAT) that have a few hundred copies, and ensuring that the CATs are coated with appropriate reagents to generate nanospheres that have a uniform size around 250 nm with a distribution around 225-275 nm.

The method, in one embodiment further comprises: distributing the CATs on a bio-chip in a densely packed collection and attaching them to the surface with removal of the coating materials, and ensuring that the CATs remain bound to the slide through multiple cycles of sequencing reactions.

In some embodiments, the target biomolecules are detected and/or sequenced and authenticated based on repeat hybridizations. This facilitates improved accuracy, including a decrease in sensitivity and/or specificity to provide improved target identification and/or sequencing.

In some embodiments, single base extension assays and oligonucleotide ligation assays are performed at single molecule levels to provide authentication. This level of authentication allows very high multiplexing and digital counting to quantify relative and absolute abundance with a higher accuracy previously unavailable via optical imaging.

### Sequencing

Optical detection imaging systems are diffraction-limited, and thus have a theoretical maximum resolution of—300 nm with fluorophores typically used in sequencing. To date, the best sequencing Systems have had center-to-center spacings between adjacent polynucleotides of—600 nm on their arrays, or—2× the diffraction limit. This factor of 2× is needed to account for intensity, array & biology variations that can result in errors in position. To achieve a 10 genome, an approximately 200 nm center to center spacing is required, which requires sub-diffraction-limited imaging capability.

For sequencing, the purpose of the system and methods described herein are to resolve polynucleotides that are sequenced on a substrate with a center-to-center spacing below the diffraction limit of the optical system.

As described herein, we provide methods and systems to achieve sub-diffraction-limited imaging in part by identifying a position of each analyte with a high accuracy (e.g., 1 Onm RMS or less). By comparison, state of the art Super Resolution systems can only identify location with an accuracy down to 20 nm RMS, 2× worse than this system. Thus, the methods and system disclosed herein enable sub-diffraction limited-imaging to identify densely-packed molecules on a substrate to achieve a high data rate per unit of enzyme, data rate per unit of time, and high data accuracy. These sub-diffraction limited imaging techniques are broadly applicable to techniques using cycled detection as described herein.

### Concatemers

Creation of Circularized ssDNA Targets

In some embodiments, described herein are methods of preparing a library of concatemers (CATs) to distribute as a layer onto the surface of a substrate, e.g., as randomly distributed, densely packed layer. To synthesize concatemers comprising target DNA to be sequenced, first, target DNA can be amplified and converted into circular DNA templates. In some embodiments, amplification products undergo circular template ligation, which can be conducted via template mediated enzymatic ligation (e.g., T4 DNA ligase) or template-free ligation using special DNA ligases (i.e., CircLigase) to form a precursor to the concatemers formed via amplification of the circular DNA templates. In some embodiments the amplification is performed by rolling circle amplification. In some embodiments, the CATs may have a first dimension along the axis parallel to the substrate of nm about 1.6 to about 80. nm about 1.6 to about 2.2, about 1.6 to about 3.2, about 1.6 to about 5, about 1.6 to about 8, about 1.6 to about 20, about 1.6 to about 80, about 2.2 to about 3.2, about 2.2 to about 5, about 2.2 to about 8, about 2.2 to about 20, about 2.2 to about 80, about 3.2 to about 5, about 3.2 to about 8, about 3.2 to about 20, about 3.2 to about 80, about 5 to about 8, about 5 to about 20, about 5 to about 80, about 8 to about 20, about 8 to about 80, or about 20 to about 80. nm about 1.6, about 2.2, about 3.2, about 5, about 8, about 20, or about 80. nm at least about 1.6, about 2.2, about 3.2, about 5, about 8, or about 20. nm at most about 2.2, about 3.2, about 5, about 8, about 20, or about 80.

### Structural Rigidity of CATs

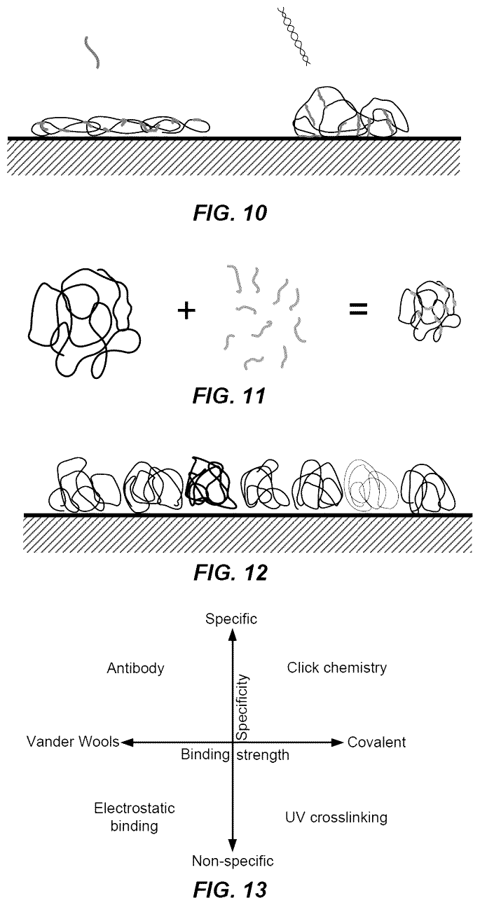

In some embodiments, especially when the surface of the substrate in the optical system is un-patterned and when the analytes comprise DNA concatemers, the DNA concatemers do not comprise a rigid structure. The structure is mutable and increases in first dimension along the axis parallel to the substrate over time as seen in FIG. 10 (left). The increase in first dimension along the axis parallel to the substrate disrupts sub-diffraction limited imaging capability. Thus, disclose herein are methods for generating a rigid structure of the analytes.

To achieve structural rigidity, in some embodiments the analyte is bonded to a support which is immobilized onto a substrate. Such solid support could be spherical or circular. Support can be provided by, for example, a nucleic acid origami structure, a metal or non-metal nanoball, or circular disks to which one side of the analyte binds. In some embodiments the nucleic acid origami structure is comprised of a nucleic acid molecule which could include DNA. In some embodiments such DNA origami structures serving as solid support for analytes are treated with covalent crosslinking in order to withstand changes associated with sequencing related temperature cycling. In some embodiments, structural support is provided by a carbon nanoball which has been functionalized with primers that bind concatemers which can be loaded to the substrate surface.

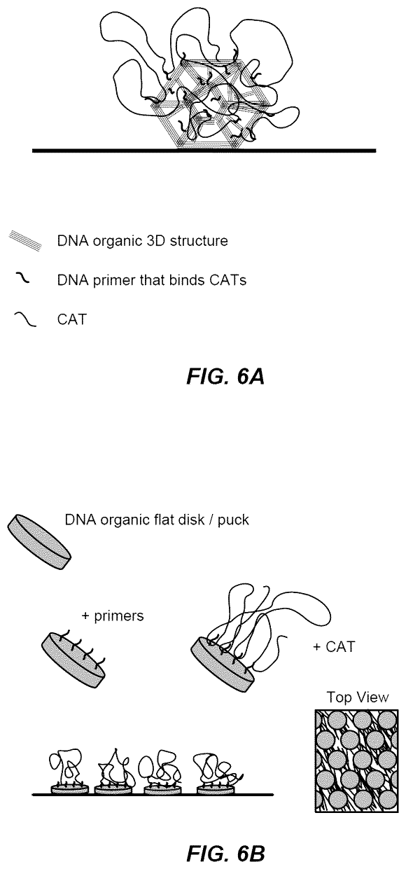

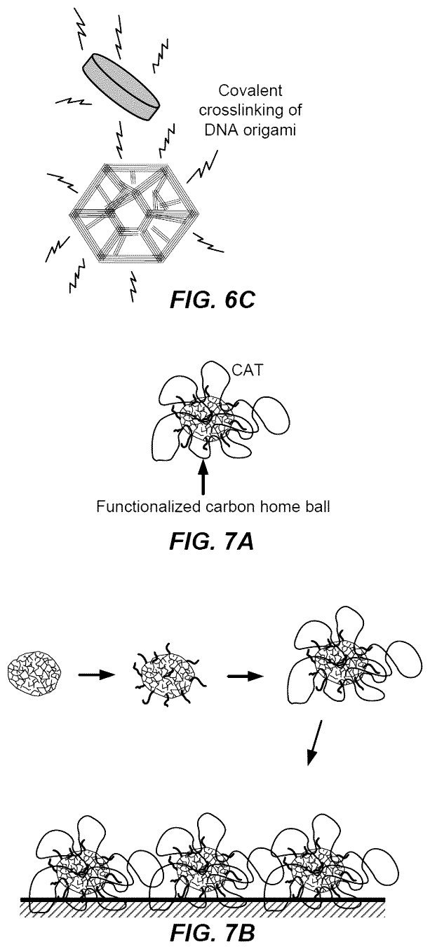

Examples of DNA origami structures are depicted in FIGS. 6A-6C. In one example, staple strands hybridize to CAT and fold it into a condensed shape. The staple strands may be modified or unmodified. The staple strands may be dye labeled. The staple strands may hybridize to primer sequences on the CAT. The staple sites may serve as initiation sites for single-strand binding proteins or “hair growth” as described herein. The CAT may remain folded throughout the sequencing process. The folded CAT may remain close to its initial size throughout the sequencing process.

In another example, as depicted in FIG. 6A, the DNA origami serves as a support structure for the CAT. The DNA origami may contain primers designed to bind to the CAT. The DNA origami and the CAT may be combined in solution, and then the combined CAT-DNA origami structure loaded onto the surface. The DNA origami may be loaded on the surface, then contacted with the CAT. In one example, the DNA origami may be a flat disk, block or a puck, as depicted in FIG. 6B. One site of the DNA origami may contain primers to bind to the CAT. One side of the DNA origami may be functionalized to bind to a surface. In one example, the DNA origami structures are covalently crosslinked, such as depicted in FIG. 6C. In some embodiments, the covalently crosslinked DNA origami structures are resistant to sequencing-related changes in temperature.

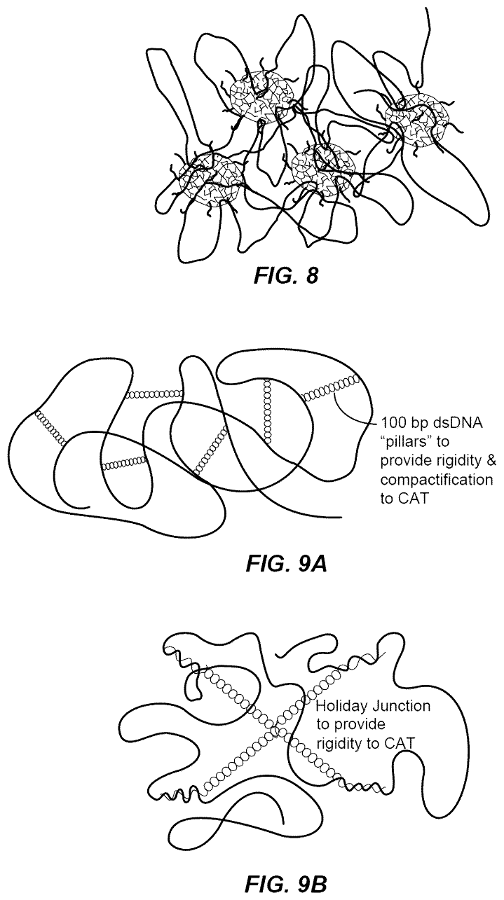

In some embodiments the plurality of support is provided by metal or non-metal carbon nanoballs. In some embodiments the plurality of structural support is provided by proteins functionalized with linkers. Some examples of metal and non-metal carbon nanoballs are depicted in FIGS. 7A-8. In one example, a carbon nanoball is functionalized with primers that bind CATs, as depicted in FIG. 7A. A carbon nanoball may be functionalized with primers. The functionalized carbon nanoball may bind to and serve as a support structure to a CAT. The functionalized carbon nanoball may bind to and serve as a support structure to a plurality of CATs. The functionalized carbon nanoball may be loaded on a surface following binding of the CAT, as depicted in FIG. 7B. A single CAT may be stabilized on a plurality of supports. One example of this is depicted in FIG. 8.

In some embodiments, a solid-support is provided during rolling circle amplification. An example of this is depicted in FIG. 19A. As the CAT is synthesized, the ssDNA may be wrapped around a solid support. The solid support may attract the CAT by electrostatic force. The solid support may comprise a positively charged surface, a carbon nanotube, or a gold nanoparticle. The solid support may have a primer for CAT generation attached.

In another example, the solid support comprises a nanotube or nanoparticles functionalized with primers. The primers may hybridize to CATs to provide a surface for the CAT molecule to attach. FIG. 19B depicts one example of CAT folding on a nanotube after CAT synthesis.

To provide rigidity and compact ability to analytes including concatemers, in some embodiments the support structure is comprised of linkers which bind to one or more analyte. In some embodiments, the linkers are, for example nucleic acid primers. In some embodiments, likers are configured to bind repeating regions of analytes. Such linkers can comprise nucleic acid molecules including DNA or RNA, either of which could be single or double stranded. In some embodiments, the DNA or RNA linkers could have known sequences, comprising a barcode sequence, an adaptor sequence, a primer sequence, or any combination thereof.

In some embodiments, structural support is provided by 100 base pair (bp) stretches of double stranded DNA serve as scaffolding for concatemers. In some embodiments, the double stranded DNA is a length of 30 nm. In some embodiments, the double-stranded DNA is a length of at least about 1 nm, about 2 nm, about 3 nm, about 4 nm, about 5 nm, about 6 nm, about 7 nm, about 8 nm, about 9 nm, about 10 nm, about 11 nm, about 12 nm, about 13 nm about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 39 nm, or about 40 nm. An example is depicted in FIG. 9A. In some embodiments, double stranded DNA scaffolding comes in the form of a Holliday junctions. An example of a CAT stabilized with a Holliday junction is depicted in FIG. 9B. FIG. 10 depicts an example of compactification with ssDNA (left panel) and double-stranded DNA (right panel). In this example, the CAT with dsDNA supports has increased structure and is more compact. In another example, structure and/or rigidity is added to the CATs using single-stranded primers. An example is depicted in FIG. 11.

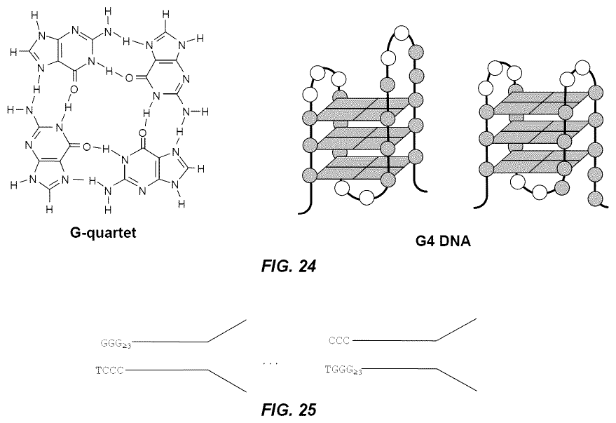

In some embodiments, structural support and organization of CATs is provided by the introduction of G4 motifs (G_{≥3}N×G_{≥3}N×G_{≥3}N×G_{≥3}), which condense the CATs, allow for more molecules per micron, and bolster each CAT off the flow cell surface. In some embodiments, the G4 motif is a G4 motif as depicted in FIG. 24. A G4 motif may be introduced using a modified Y-shaped adaptor, an example of which is depicted in FIG. 25. The G4 sequence may be placed practically anywhere in the stem or single-stranded portion of the adapter. In some embodiments, following ligation, library molecules would be circularized and converted into CATs, containing a plurality of G4 motifs. In some embodiments, G4 DNA is generated by addition of a salt solution. In some embodiments, G4 DNA is generated by heating and cooling the CAT to an optimal temperature.

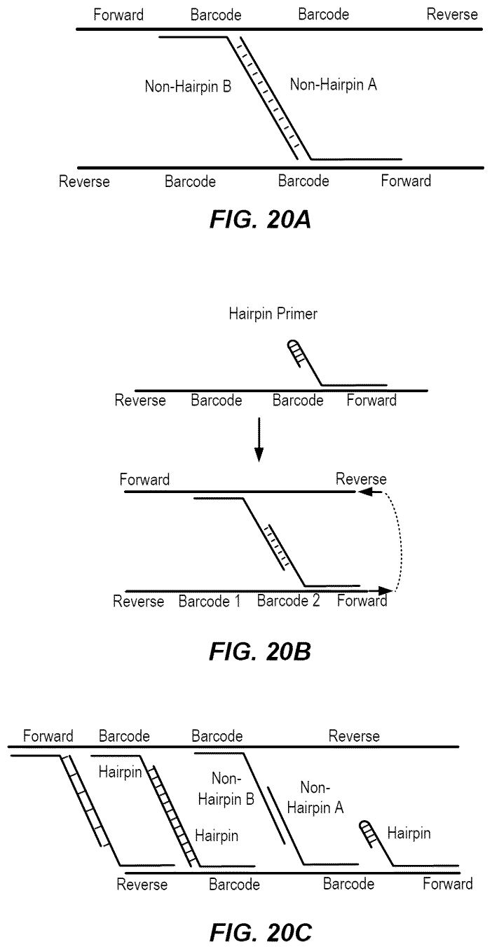

In some embodiments, structural rigidity is provided by a combination of hairpin and non-hairpin primers. In some embodiments, DNA hairpins or complementary DNA sequences are used to link together multiple adaptor regions within a sequencing library. The multiple adaptor regions may be present on a single CAT or on multiple CATs. In some embodiments, the multiple adaptor regions are used to link a single CAT. An example of this process is depicted in FIGS. 20A-20C. In one embodiment, after mixing and annealing, a 5′ tail comprised of either a palindromic hairpin or two complementary DNA sequences are mixed with the DNA library prior to loading onto a flowcell, as depicted in FIG. 20A. Without being bound by theory, these staples reduce the physical size the DNA library by linking together adjacent or non-adjacent copies of the adaptor regions. In some embodiments, the 3′ end can be blocked, reversible blocked, or a free 3′OH. In some embodiments, a 5′ palindromic “hairpin” primer in which the 3′ end hybridizes to one or more locations on the sequencing library adaptor region. In some embodiments, a 5′ palindromic region hybridizes with a secondary primer and provides structural rigidity to reduce physical size of the loaded DNA sample. In some embodiments, the 3′ end can hybridize to any adaptor region including: forward sequencing primer, reverse sequencing primer, any barcode primer regions, or any combination of the previous.

One embodiment of this process is depicted in FIG. 20B. In some embodiments, the set of two or more “non-hairpin” primers (Primer A and Primer B) in which Primer A contains a 5′ tail and Primer B contains the complement of the 5′ tail of Primer 1. In some embodiments, the 5′ region hybridizes with the 5′ tail of the complement primer and provides structural rigidity to reduce physical size of the loaded DNA sample. In some embodiments, the 3′ prime end can hybridize to one or more locations on the sequencing library adaptor region including: forward sequencing primer, reverse sequencing primer, any barcode primer regions, or any combination of the previous.

In some embodiments, the 5′ Hairpin or non-hairpin region can be universal, in which any 3′ primer has a 5′ hairpin or non-hairpin that can hybridize to the 5′ hairpin or non-hairpin of any other primer regardless of location on the library adaptor (5′ Hairpin-Forward Sequencing Primer can hybridize to 5′Hairpin-Reverse Sequencing Primer). An example of universal primers is depicted in FIG. 20C. In some embodiments, the 5′ hairpin or 5′ non-hairpin region can be unique, in which the 3′ primer can only hybridize to a second primer which hybridizes to the same location on the library adaptor (Hairpin1—Forward can only hybridize with Forward, Hairpin2—Reverse can only hybridize with Reverse). In some embodiments, the 5′ palindromic (hairpin) or 5′ non-hairpin primer contains a reversible 3′ block that prevents enzymatic incorporation. In some embodiments, the 3′ Block can include Phosphate groups, disulfide, azidomethyl, amino (ONH2) . . . .

In some embodiments external structure, support or stability is provided by in the form of concatemer crowding. In some embodiments, external support in the form of crowding is achieved through the use of dark CATs. Dark CATs lack sequencing primers and thus do not show up in sequencing imaging experiments, however they prevent the sequencing CATs from taking up too much space, moving around, or losing their structural integrity. In some embodiments dark CATs are themselves also supported by DNA origami or other structural additions.

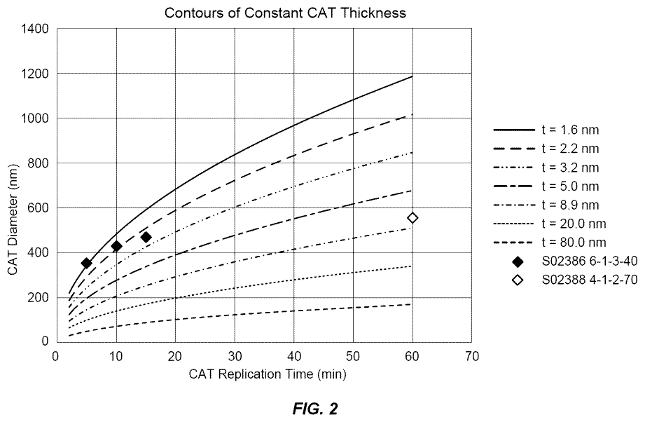

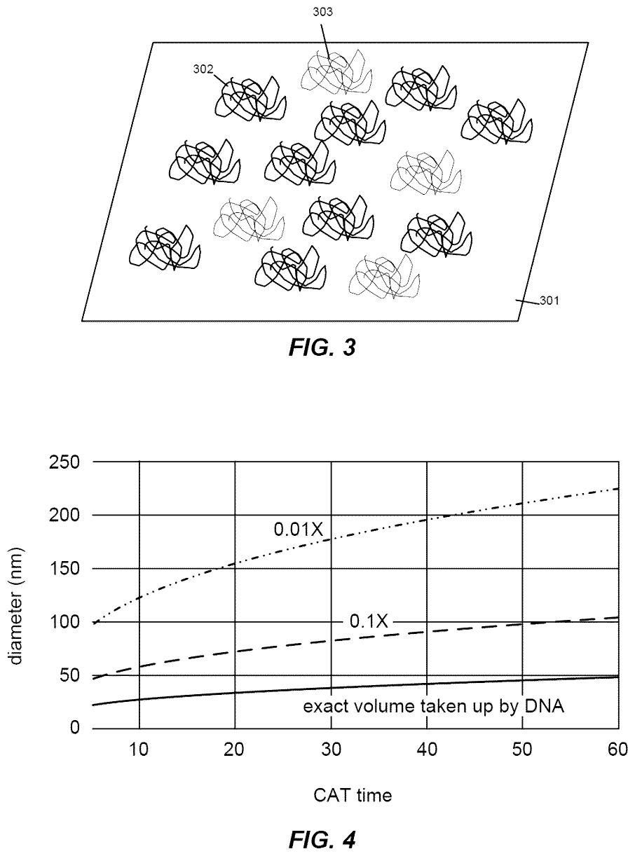

One embodiment of the use of dark CATs is depicted in FIG. 3. A surface 301 may be covered with dark CATs 302 and sequencing CATs 303. In some embodiments, the dark CATs and the sequencing CATs are present in equal amounts. In some embodiments, there are more dark CATs than sequencing CATs. In some embodiments there are more sequencing CATs than dark CATs. A side view of the use of dark CATs is depicted in FIG. 12.

In some embodiments the analyte is bound to one solid support, in some embodiments the analyte is bond to a plurality of solid supports. In some embodiments the plurality of support is provided by DNA origami structures.

In some embodiments the method of imaging involves adding artifacts which increase steric hinderance. In some embodiments this is achieved through the addition of covalent bonding between the DNA primers and a polymer or biomolecule. In some embodiments, this biomolecule is, for example, a peptide, sugar molecule, PEG or detergent. In some embodiments, these molecules have additional functionality, such as a pH change sensitive peptide that switches from a relaxed to a condensed state based on pH changes. In some such embodiments, a CAT can be condensed by inducing the conformation change of the crosslinker molecules. In some embodiments, non-nucleic acid, biopolymer based crosslinkers also contain a reactive group that links two crosslinkers together. In some embodiments such crosslinking is achieved via the use of a cystine amino acid that specifically reacts only with other free cystines to form a covalent bond. In some embodiments, multiple reactive crosslinkers are used to achieve a compacted state.

### RCA/RCR Basic Technique

Rolling circle replication describes a process of unidirectional nucleic acid replication that can rapidly synthesize multiple copies of circular molecules of DNA or RNA.

RCA (rolling circle amplification) is an isothermal nucleic acid amplification technique where the polymerase continuously adds single nucleotides to a primer annealed to a circular template which results in a long concatemer ssDNA that contains tens to hundreds of tandem repeats (complementary to the circular template).

Rolling circle amplification can be performed by exposing the circular DNA templates to: 1. A DNA polymerase. 2. A suitable buffer that is compatible with the polymerase. 3. A short DNA or RNA primer. 4. Deoxynucleotide triphosphates (dNTPs).

In some embodiments, the polymerase used in rolling circle amplification is Phi29, Bst, or Vent exo-DNA polymerase for DNA amplification, and T7 RNA polymerase for RNA amplification. RCA can be conducted at a constant temperature (room temperature to 37° C.) in both free solution and on top of deposited targets (solid phase amplification). A DNA RCA reaction typically proceeds via primer-induced single-strand DNA elongation.

In some embodiments, a method for constructing concatemer libraries of sequencing substrates to load onto a physical substrate, such as a flow cell. In some embodiments, concatemer libraries of sequencing substrates are where ‘hairs’ of ssDNA molecules which can be generated by using a reverse primer to synthesize in the opposite direction as the extending concatemer DNA. These ‘hairs’ can be used to control the size and/or exclusion properties of the concatemers. In some embodiments, the sequencing reaction described herein occurs using the ssDNA ‘hairs’ as templates.

### Terminating RCR Reaction

The rolling circle amplification of the CAT can be stopped by the addition of EDTA to chelate the essential Mg2+ co-factor of the phi29 enzyme. Phi29 is a strongly displacing polymerase, while the standard polymerases used for sequencing, for example Therminator 9, are only weakly displacing. A more displacing enzyme for sequencing this substrate may be used or adapted.

Alternatively, one may use single strand binding proteins (SSBs) or helicases, or combinations of them to aid in the displacement. These may be added to the extension reaction or used as pre-incubation operations to prepare the substrate for sequencing.

Alternatively, the rolling circle reaction may be stopped using an unlabeled reversible terminator. This may be a way to make the stoppage more uniform within the solution, yielding more uniform-sized CATs than stoppage with EDTA. Additionally, the sequencing reaction may then be initiated from the unblocking operation, followed by extension with labeled reversible terminator nucleotides. This may allow for the natural selection of substrates that where the extending 3′ end was accessible for the normal reactions of sequencing by synthesis.

The phi29 is likely very tightly bound to the extending end of the CAT. The use of a reversible terminator to stop the reaction may destabilize that interaction. Other protein denaturants like chaotropic salts or detergents may be necessary to displace the phi29 to enable the sequencing reaction

### Concatemer Composition

The CATs have several identical copies of the target DNA on the extending single strand. CATs can also have several identical reverse copies of the target DNA on ssDNA ‘hairs’ generated as described above.

In some embodiments concatemers are at least 1,000 nucleotides in length (no more than, from 400,000).

In some embodiments, concatemers are at least 150 nm in diameter (no more than 300 nm). Preferably, the exclusion zone between adjacent concatemers is not less than the minimum center-to-center distance necessary to achieve the desired density or pitch.

### Densely-Packed Random Arrays

### Methods of Making Arrays (Randomly Distributed Close Packed Layer of Concatemers)

### Controlled Spacing

Provided herein are several mechanisms to control the distribution of minimum center-to-center distance between CATs arrayed on an un-patterned surface. In some embodiments, these methods and compositions facilitate formation of a uniform, close-packed self-assembled random layer of CATs with a controlled minimum center-to-center distance between adjacent CATs such that they can be sequenced with minimal cross-talk between the dye-labeled sequencing substrates.

The CATs themselves are mutually repellant in solution due to their strong negative charge, but they may nonetheless be too close to each other for effective diffusion-limited resolution of labeled adjacent CATs once adsorbed to a surface.

In some embodiments, the concatemers are ‘encased’ or ‘enveloped’ in a shell of a repellant or attractive substance to increase their effective exclusion size without altering the size of the CAT itself or the number of copies of the sequencing substrate they contain.

In some embodiments, a protein layer to which the CATs adsorb on the surface of the substrate is modified to space the interacting proteins out on the surface. For example, the CATs can interact with the glass, silicon or modified (e.g. amino-silanated) surface through an interaction with proteins that have been previously adsorbed to the surface.

Thus, modifications of the CAT or the protein partner of the binding pair can assist in size exclusion to achieve a uniform, densely-packed layer of concatemers on a surface without specific attachment points for the CATs. In some embodiments, these modifications include crosslinking or attaching molecules like PEG or polysaccharide to coat the CAT or its protein binding partner.

The inner core in this embodiment may be multiple copies of a DNA target that are entwined. The outer layer, i.e., the coating, can include compounds like PEG, compounds with zwitterionic features, ampholine ampholytes, sulphobetaine, and other similar molecules with the positive charges interacting with nucleic acid on the inside and negative charges on the outside the ensure the nanospheres do not clump.

### Loading of CATs on the Chip

Different methods may be used to attach CATs to the surface of the chip. As depicted in FIG. 13, methods of attaching CATs to the surface of the chip vary based on whether the bidnign is specific or non-specific and on binding strength. FIG. 13 depicts non-exhaustive examples of different methods of attaching CATs to the surface of the chip. In some embodiments, the CATS are attached through antibody binding, electrostatic binding, click chemistry, or UV-crosslinked.

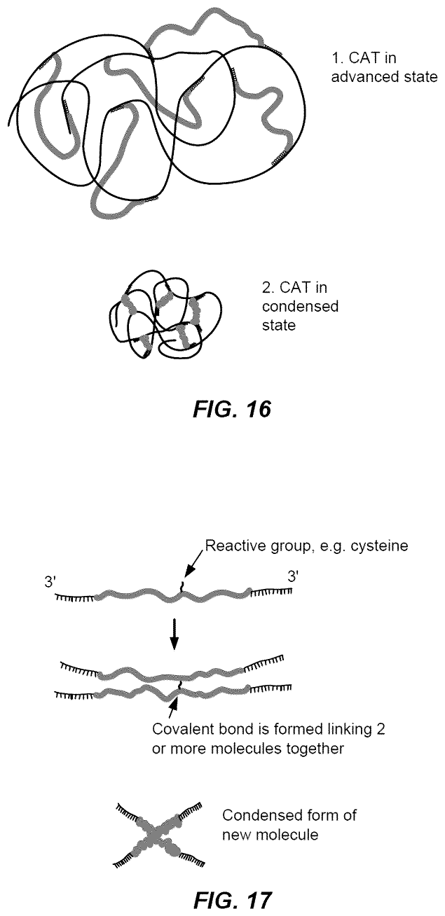

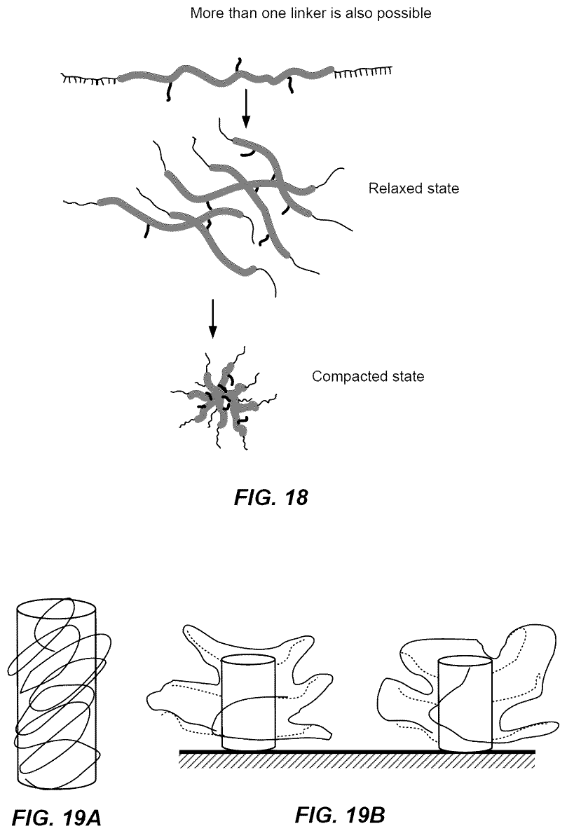

In some embodiments, the surface is functionalized with a biopolymer. In some embodiments, the biopolymer comprises a binding moiety. In some embodiments, the binding moiety is streptavidin. In some embodiments, the analyte is linked to the binding moiety via a linker as described herein. In some embodiments, the linker attaches to the analyte via DNA hybridization. The linker may be a molecule as described in FIGS. 15-18. In one example, the

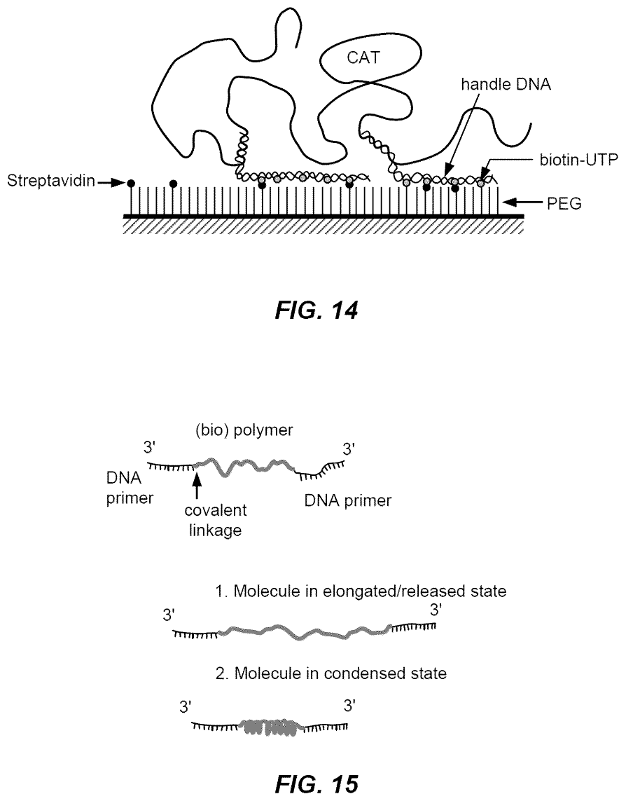

In one embodiment, the surface is functionalized with PEG. The PEG may comprise streptavidin. The analyte may be attached to a linker. The linker may comprise a biopolymer. The linker may comprise nucleic acid. The linker may comprise DNA. The linker may comprise biotin. The biotin may bind to streptavidin, holding the analyte at the surface. An example is depicted in FIG. 14.

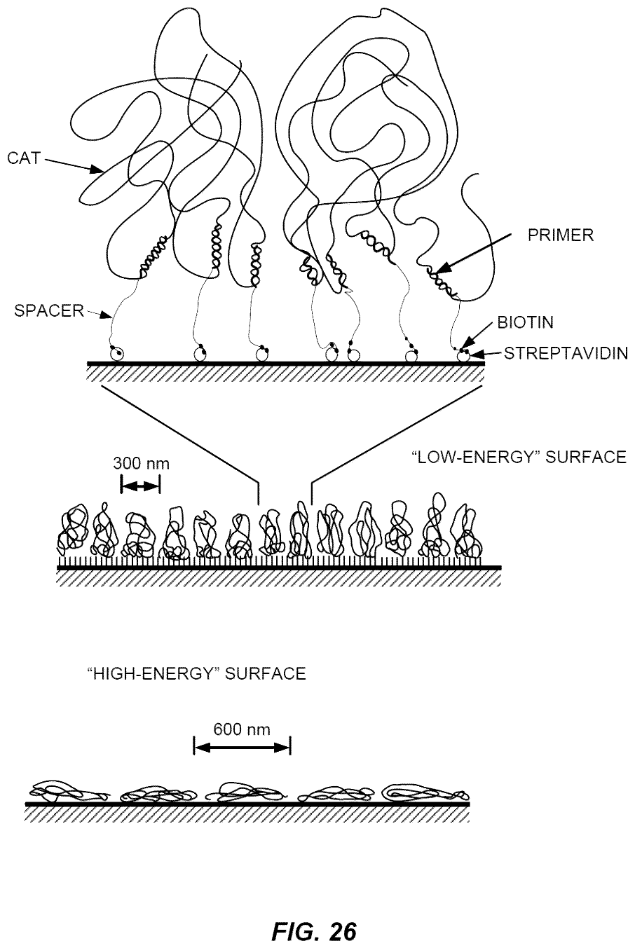

In one embodiment, the surface is functionalized with streptavidin. The streptavidin may bind to a linker. The linker may comprise biotin. The linker may comprise an analyte-binding end. The analyte-binding end may comprise DNA. The analyte-binding end may comprise a primer. An example is depicted in FIG. 26.

In some embodiments concatemers are distributed onto an unpatterned surface of a substrate in a high density layer. This close-packed formation facilitates formation of tightly packed sequencing substrates to enable higher throughput and/or lower cost sequencing. In some embodiments, said surface is patterned

In some embodiments, concatemers are loaded on a biochip and closely packed to enable a center to center distance of—250 nm with a variance of +/−25 nm.

In some embodiments, the average center-to-center distance between molecules of about 315 nm. In some embodiments, the plurality of analytes (e.g., nucleic acid molecules) may be deposited adjacent to a surface such that adjacent analytes of the plurality of analytes may have average center-to-center spacings of at least 10 nanometers (nm), 50 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, or more. The average center-to-center spacings may be less than or equal to 500 nm, 490 nm, 480 nm, 470 nm, 460 nm, 450 nm, 440 nm, 430 nm, 420 nm, 410 nm, 400 nm, 390 nm, 380 nm, 370 nm, 360 nm, 350 nm, 340 nm, 330 nm, 320 nm, 310 nm, 300 nm, 290 nm, 280 nm, 270 nm, 260 nm, 250 nm, 240 nm, 230 nm, 220 nm, 210 nm, 200 nm, 190 nm, 180 nm, 170 nm, 160 nm, 150 nm, 140 nm, 130 nm, 120 nm, 110 nm, 100 nm, 50 nm, or less.

In some embodiments, the concatemers comprise a coating to achieve a lower threshold of center-to-center distances between adjacent concatemers to minimize crosstalk during detection. In some embodiments, after binding the concatemers to the surface, the coating is dissolved and the CATs attached to the surface and can be sequenced.

Another protein such as BSA may be used, either by chemically crosslinking to the CAT or the protein binding partner, or by attaching the spacer protein (e.g. BSA) to an oligonucleotide complementary to the common library adapter sequence through strepavidin interaction. Using BSA to coat the CAT may have the additional benefit of making a protein gel in the bound layer of CATs which may make the local environment for the enzymatic reaction more similar to the natural environment of the nucleus where polymerases normally act.

One may also be able to hybridize long single stranded oligonucleotides that are partially complementary to the common library adapter sequence and extend beyond that sequence without homology. In some embodiments, the long single stranded oligonucleotides are the hairs mentioned above in Paragraph [0096]. Such long oligonucleotides may act to increase the size of the CAT without altering the number of sequencing substrates it contains. After surface attachment, these long oligonucleotides may be washed away, and each CAT may collapse towards the center of its attachment site, increasing the effective center to center distance between adjacent CATs.

DNA may also be used to modify the protein binding partner (by crosslinking or attachments such as strep-avidin) to create a surface that has attractive protein binding sites separated by repellant areas, for instance due to their negative charge.

Deposition of a Closely-Packed Concatemer Layer onto an Unpatterned Surface

One of the limitations to optimum packing density of biological analytes from an aqueous solution onto an un-patterned, adherent solid surface is that the random binding of the analytes onto the surface does not provide for maximal close-packing due to the inability of the adhered analyte to move laterally and minimize spacing between bound molecules. As a result, this random irreversible sticking of analytes produces spacing defects in what may otherwise be arranged into a maximally close-packed array.

However, many biological analytes, including proteins and nucleic acids, are known to be surface active and migrate to the air-water interface that results in a lowering of the surface tension at that interface, to produce a metastable monolayer of biomolecules. In this case, the surface-active analytes are free to move laterally at the interface and achieve a maximal close-packed density, with unfavorable hydrophobic interactions in solution being the driving force for maximal packing.

Therefore, in some embodiments, close-packed, spontaneously formed monolayer constructs of biomolecules at the air-water interface can be transferred or deposited onto a solid surface by pulling or dragging a bolus of the biomolecule solution across the solid surface that is already in contact with air. Thereby, the close-packed biomolecule construct at the air-water interface is deposited onto the solid surface from the point of three-phase (air-water solid) contact as the bolus moves across the solid surface.

In some embodiments, a protein layer may be laid down on the surface before the CATs are added. Then the CATs may be added to the already laid down protein layer. This sequential addition may be particularly effective if the binding protein is the modified partner. In some embodiments, the protein comprises streptavidin.

### Surface Passivation

In some embodiments, the surface can be chemically modified to provide for a low energy surface whereby spreading of the template can be controlled. Without being bound by theory, the advantages of this control include high yield per unit area, high accuracy of sequence read, and potentially longer reads.

Described herein are methods for lowering the surface energy during template immobilization and thereby controlling template spreading to provide for compact, high density arrays for sequencing. Methods include chemical surface hydrophobation by reaction of the surface with hydrophobic chemical reagents, including both covalent and non-covalent attachment strategies.

In some embodiments, chemical modification of the surface can be used to lower surface energy and control spreading through the introduction of hydrophobic moieties to the surface prior to DNA immobilization. In some embodiments, chemical modification comprises covalent attachment of reactive molecules. In some embodiments, the reaction molecules comprise medium to long alkane chains. In some embodiments, the reactive molecules comprise fluorine groups. In some embodiments, chemical modification comprises attachment of surface active agents comprising hydrophobic groups. In some embodiments, the surface active agents comprise alkyl surfactants, fluorinated surfactants, or block copolymers. In some embodiments, the surface active agents comprise a peptide. In some embodiments, the surface active agents comprise streptavidin.

Alternatively, the surface can be modified by contacting the surface with surface coating molecules. As defined herein, a “surface-coating molecule” comprises a molecule that contacts the surface. The surface coating molecule may chemically modify the surface. The surface-coating molecule may lower the surface energy of the surface. The surface-coating molecule may control spreading of the CATs once attached to the surface. In some embodiments, the surface-coating molecule comprises one or more hydrophobic moieties. In some embodiments, the surface-coating molecule comprises one or more negatively charged moieties. In some embodiments, the one or more hydrophobic moieties comprise alkane chains comprising at least 6 carbons. In some embodiments, the one or more hydrophobic moieties comprise alkane chains comprising at least 12 carbons. In some embodiments, the one or more hydrophobic moieties comprises a plurality of fluorine groups. In some embodiments, the surface-coating molecule comprise streptavidin. In some embodiments, the surface-coating molecule are coupled to said surface. In some embodiments, the d surface-coating molecule are covalently attached to said surface. In some embodiments, the surface-coating molecule are in solution. In some embodiments, the one or more hydrophobic moieties comprise a block copolymer. In some embodiments, the said block copolymer is poloxamer. In some embodiments, the surface-coating molecule are contacted to said surface before providing said one or more analytes. In some embodiments, the surface-coating molecule are contacted to said surface contemporaneously with providing said one or more analytes.

### Sequencing

### Sequencing Work Flow

In some embodiments, provided herein are methods to detect the sequences of polynucleotides from the concatemers, e.g., through forming a densely-packed layer on an unpatterned surface and performing cycled sequencing by synthesis. In some embodiments, said surface is patterned.

The detection of targets and their authentication based on repeat hybridizations is a key feature enabling target identification and counting for quantification.

Syncing and Signal Calling (ddNTP Capping of Unreacted Oligonucleotides)

In some embodiments, the sequencing by synthesis includes addition of an irreversible ddNTP terminator after an extension cycle to cap unextended oligonucleotides. For example, after getting maximal initiation and/or extension with mixture of labeled and cold reversible terminators, a cycle of extension (e.g., with a different polymerase that can, better incorporate ddNTPs) and very high concentrations of all four ddNTPs. This operation may irreversibly terminate the extension of any sequencing template within a CAT that failed to extend at the cycle in question. Although this may lead to progressive loss of signal, proportional to the inefficiency of initiation or extension, importantly it may also reduce background at subsequent cycles of those templates within the CAT that ‘skipped’ extension at any cycle, a process which results in mixed signal from lagging synthesis on some of the identical templates within the CAT.

This process may lead to increased synchronization of templates within a CAT, yielding less signal from lagging templates, so purer signal from the correct base in the sequence. All other things being equal, it may lead to longer effective sequence reads.

### Reaction

The CATs have several identical copies of the target DNA, but the last copy made during rolling circle amplification is unique in that it contains an actively extending 3′ end. This ssCircle and its actively extending end are likely to be near the center of the ball of DNA that is the CAT, so it is near the center of the exclusion zone within the monolayer of CATs. It is also away from the surface on which that monolayer is formed. Raising the actively extending end away from the surface may increase the accessibility for the chemicals and enzymes used in the sequencing reaction, and also perhaps raise the dye labels above the focal plane of background fluorescence on the surface. These properties make it ideal for single-molecule sequencing.

### Paired End Sequencing and Unique Molecule Identifiers

Unique Molecular identifiers (UMIs) have been used to tag molecules to enable identification of duplicate PCR products and also to enable double stranded sequencing applications that reduce error.

In some embodiments, adapters that contain UMIs are incorporated into the circularized DNA template used to form the concatemer.

In one embodiment, UMI A1 and A2 adaptors are added to the 5′ and 3′ ends of Strand A and B. A1 and A2 can have barcodes for sample ID. They also have regions used for ligation/circle generation and sequencing primer binding regions to enable sequencing both strands. The adaptors may also have the UMI sequences.

After the completion of sequencing the UMIs can be used to locate circles emanating from the same DNA fragment and analyzed as paired end reads. Paired end reads are useful for mapping if the read lengths are short.

Although UMI may be used, many applications, such as NIPT, PCR amplified panels, and large portions of the genome can be reliably sequenced without having paired end capability.

### Imaging and Cycled Detection

As described herein, each of the detection methods and systems required cycled detection to achieve sub-diffraction limited imaging. Cycled detection includes the binding and imaging or probes, such as antibodies or nucleotides, bound to detectable labels that can emit a visible light optical signal. By using positional information from a series of images of a field from different cycles, deconvolution to resolve signals from densely packed substrates can be used effectively to identify individual optical signals from signals obscured due to the diffraction limit of optical imaging. After multiple cycles the precise location of the molecule may become increasingly more accurate. Using this information additional calculations can be performed to aid in crosstalk correction regarding known asymmetries in the crosstalk matrix occurring due to pixel discretization effects.

### Methods for Optical Detection of Analytes

In some embodiments, optical signals are digitized, and analytes are identified based on a code (ID code) of digital signals for each analyte.

As described herein, analytes are deposited to a solid substrate, and probes are bound to the analytes. Each of the probes comprises tags and specifically binds to a target analyte. In some embodiments, the tags are fluorescent molecules that emit the same fluorescent color, and the signals for additional fluors are detected at each subsequent pass. During a pass, a set of probes comprising tags are contacted with the substrate allowing them to bind to their targets. An image of the substrate is captured, and the detectable signals are analyzed from the image obtained after each pass. The information about the presence and/or absence of detectable signals is recorded for each detected position (e.g., target analyte) on the substrate.

In some embodiments, the present disclosure comprises methods that include operations for detecting optical signals emitted from the probes comprising tags, counting the signals emitted during multiple passes and/or multiple cycles at various positions on the substrate, and analyzing the signals as digital information using a K-bit based calculation to identify each target analyte on the substrate. Error correction can be used to account for errors in the optically-detected signals, as described below.

In some embodiments, a substrate is bound with analytes comprising N target analytes. To detect N target analytes, M cycles of probe binding and signal detection are chosen. Each of the M cycles includes 1 or more passes, and each pass includes N sets of probes, such that each set of probes specifically binds to one of the N target analytes. In certain embodiments, there are N sets of probes for the N target analytes.

In each cycle, there is a predetermined order for introducing the sets of probes for each pass. In some embodiments, the predetermined order for the sets of probes is a randomized order. In other embodiments, the predetermined order for the sets of probes is a non-randomized order. In one embodiment, the non-random order can be chosen by a computer processor. The predetermined order is represented in a key for each target analyte. A key is generated that includes the order of the sets of probes, and the order of the probes is digitized in a code to identify each of the target analytes.

In some embodiments, each set of ordered probes is associated with a distinct tag for detecting the target analyte, and the number of distinct tags is less than the number of N target analytes. In that case, each N target analyte is matched with a sequence of M tags for the M cycles. The ordered sequence of tags is associated with the target analyte as an identifying code.

### Quantification of Optically-Detected Probes

After the detection process, the signals from each probe pool are counted, and the presence or absence of a signal and the color of the signal can be recorded for each position on the substrate.

From the detectable signals, K bits of information are obtained in each of M cycles for the N distinct target analytes. The K bits of information are used to determine L total bits of information, such that K×M=L bits of information and L≥log 2 (N). The L bits of information are used to determine the identity (and presence) of N distinct target analytes. If only one cycle (M=1) is performed, then K×1=L. However, multiple cycles (M>1) can be performed to generate more total bits of information L per analyte. Each subsequent cycle provides additional optical signal information that is used to identify the target analyte.

In practice, errors in the signals occur, and this confounds the accuracy of the identification of target analytes. For instance, probes may bind the wrong targets (e.g., false positives) or fail to bind the correct targets (e.g., false negatives). Methods are provided, as described below, to account for errors in optical and electrical signal detection.

### Electrical Detection Methods

In other embodiments, electrical detection methods are used to detect the presence of target analytes on a substrate. Target analytes are tagged with oligonucleotide tail regions and the oligonucleotide tags are detected using ion-sensitive field-effect transistors (ISFET, or a pH sensor), which measures hydrogen ion concentrations in solution. ISFETs are described in further detail in U.S. Pat. No. 7,948,015, filed on Dec. 14, 2007, to Rothberg et al., and U.S. Publication No. 2010/0301398, filed on May 29, 2009, to Rothberg et al., which are both incorporated by reference in their entireties.

ISFETs present a sensitive and specific electrical detection system for the identification and characterization of analytes. In one embodiment, the electrical detection methods disclosed herein are carried out by a computer (e.g., a processor). The ionic concentration of a solution can be converted to a logarithmic electrical potential by an electrode of an ISFET, and the electrical output signal can be detected and measured.

ISFETs have previously been used to facilitate DNA sequencing. During the enzymatic conversion of single-stranded(ss) DNA into double-stranded DNA, hydrogen ions are released as each nucleotide is added to the DNA molecule. An ISFET detects these released hydrogen ions and can determine when a nucleotide has been added to the DNA molecule. By synchronizing the incorporation of the nucleoside triphosphate (dATP, dCTP, dGTP, and dTTP), the DNA sequence may also be determined. For example, if no electrical output signal is detected when the single-stranded DNA template is exposed to dATP's, but an electrical output signal is detected in the presence of dGTP's, the DNA sequence is composed of a complementary cytosine base at the position in question.

In one embodiment, an ISFET is used to detect a tail region of a probe and then identify corresponding target analyte. For example, a target analyte can be deposited on a substrate, such as an integrated-circuit chip that contains one or more ISFETs. When the corresponding probe (e.g., aptamer and tail region) is added and specifically binds to the target analyte, nucleotides and enzymes (polymerase) are added for transcription of the tail region. The ISFET detects the release hydrogen ions as electrical output signals and measures the change in ion concentration when the dNTP's are incorporated into the tail region. The amount of hydrogen ions released corresponds to the lengths and stops of the tail region, and this information about the tail regions can be used to differentiate among various tags.

The simplest type of tail region is one composed entirely of one homopolymeric base region. In this case, there are four possible tail regions: a poly-A tail, a poly-C tail, a poly-G tail, and a poly-T tail. However, it is often desirable to have a great diversity in tail regions.

One method of generating diversity in tail regions is by providing stop bases within a homopolymeric base region of a tail region. A stop base is a portion of a tail region comprising at least one nucleotide adjacent to a homopolymeric base region, such that the at least one nucleotide is composed of a base that is distinct from the bases within the homopolymeric base region. In one embodiment, the stop base is one nucleotide. In other embodiments, the stop base comprises a plurality of nucleotides. Generally, the stop base is flanked by two homopolymeric base regions. In an embodiment, the two homopolymeric base regions flanking a stop base are composed of the same base. In another embodiment, the two homopolymeric base regions are composed of two different bases. In another embodiment, the tail region contains more than one stop base.

In one example, an ISFET can detect a minimum threshold number of 100 hydrogen ions. Target Analyte 1 is bound to a composition with a tail region composed of a 100-nucleotide poly-A tail, followed by one cytosine base, followed by another 100-nucleotide poly-A tail, for a tail region length total of 201 nucleotides. Target Analyte 2 is bound to a composition with a tail region composed of a 200-nucleotide poly-A tail. Upon the addition of dTTP's and under conditions conducive to polynucleotide synthesis, synthesis on the tail region associated with Target Analyte 1 may release 100 hydrogen ions, which can be distinguished from polynucleotide synthesis on the tail region associated with Target Analyte 2, which may release 200 hydrogen ions. The ISFET may detect a different electrical output signal for each tail region. Furthermore, if dGTP's are added, followed by more dTTP's, the tail region associated with Target Analyte 1 may then release one, then 100 more hydrogen ions due to further polynucleotide synthesis. The distinct electrical output signals generated from the addition of specific nucleoside triphosphates based on tail region compositions allow the ISFET to detect hydrogen ions from each of the tail regions, and that information can be used to identify the tail regions and their corresponding target analytes.

Various lengths of the homopolymeric base regions, stop bases, and combinations thereof can be used to uniquely tag each analyte in a sample. Additional description about electrical detection of aptamers and tail regions to identify target analytes in a substrate are described in U.S. Provisional Application No. 61/868,988, which is incorporated by reference in its entirety.

In other embodiments, antibodies are used as probes in the electrical detection method described above. The antibodies may be primary or secondary antibodies that bind via a linker region to an oligonucleotide tail region that acts as tag.

These electrical detection methods can be used for the simultaneous detection of hundreds (or even thousands) of distinct target analytes. Each target analyte can be associated with a digital identifier, such that the number of distinct digital identifiers is proportional to the number of distinct target analytes in a sample. The identifier may be represented by a number of bits of digital information and is encoded within an ordered tail region set. Each tail region in an ordered tail region set is sequentially made to specifically bind a linker region of a probe region that is specifically bound to the target analyte. Alternatively, if the tail regions are covalently bonded to their corresponding probe regions, each tail region in an ordered tail region set is sequentially made to specifically bind a target analyte.

In one embodiment, one cycle is represented by a binding and stripping of a tail region to a linker region, such that polynucleotide synthesis occurs and releases hydrogen ions, which are detected as an electrical output signal. Thus, number of cycles for the identification of a target analyte is equal to the number of tail regions in an ordered tail region set. The number of tail regions in an ordered tail region set is dependent on the number of target analytes to be identified, as well as the total number of bits of information to be generated. In another embodiment, one cycle is represented by a tail region covalently bonded to a probe region specifically binding and being stripped from the target analyte.

The electrical output signal detected from each cycle is digitized into bits of information, so that after all cycles have been performed to bind each tail region to its corresponding linker region, the total bits of obtained digital information can be used to identify and characterize the target analyte in question. The total number of bits is dependent on a number of identification bits for identification of the target analyte, plus a number of bits for error correction. The number of bits for error correction is selected based on the desired robustness and accuracy of the electrical output signal. Generally, the number of error correction bits may be 2 or 3 times the number of identification bits.

### Decoding the Order and Identity of Detected Analytes

The probes used to detect the analytes are introduced to the substrate in an ordered manner in each cycle. A key is generated that encodes information about the order of the probes for each target analyte. The signals detected for each analyte can be digitized into bits of information. The order of the signals provides a code for identifying each analyte, which can be encoded in bits of information.

### Error-Correction Methods

In optical and electrical detection methods described above, errors can occur in binding and/or detection of signals. In some cases, the error rate can be as high as one in five (e.g., one out of five fluorescent signals is incorrect). This equates to one error in every five-cycle sequence. Actual error rates may not be as high as 20%, but error rates of a few percent are possible. In general, the error rate depends on many factors including the type of analytes in the sample and the type of probes used. In an electrical detection method, for example, a tail region may not properly bind to the corresponding probe region on an aptamer during a cycle. In an optical detection method, an antibody probe may not bind to its target or bind to the wrong target.

Additional cycles are generated to account for errors in the detected signals and to obtain additional bits of information, such as parity bits. The additional bits of information are used to correct errors using an error correcting code. In one embodiment, the error correcting code is a Reed-Solomon code, which is a non-binary cyclic code used to detect and correct errors in a system. In other embodiments, various other error correcting codes can be used. Other error correcting codes include, for example, block codes, convolution codes, Golay codes, Hamming codes, BCH codes, AN codes, Reed-Muller codes, Gappa codes, Hadamard codes, Walsh codes, Hagelbarger codes, polar codes, repetition codes, repeat-accumulate codes, erasure codes, online codes, group codes, expander codes, constant-weight codes, tornado codes, low-density parity check codes, maximum distance codes, burst error codes, luby transform codes, fountain codes, and raptor codes. See Error Control Coding, 2nd Ed., S. Lin and DJ Costello, Prentice Hall, New York, 2004. Examples are also provided below that demonstrate the method for error-correction by adding cycles and obtaining additional bits of information.

One example of a Reed-Solomon code includes a RS (15,9) code with 4-bit symbols, where n=15, k=9, s=4, and t=3, and n=2s−1 and k=n−2t, “n” being the number of symbols, “k” being the number of data symbols, “s” being the size of each symbol in bits, and “t” being the number of errors that can be corrected, and “2t” being the number of parity symbols. There are nine data symbols (k=9) and six parity symbols (2t=6). If base-X numbers are used, and X=4, then each fluorescent color is represented by two bits (0 and 1). A pair of colors may be represented by a four-bit symbol that includes two high bits and two low bits.

Since base-4 was chosen, seven probe pools, or a sequence of seven colors, are used to identify each target analyte. This sequence is represented by 3½, 4-bit symbols. The remaining 5½ data symbols are set to zero. A Reed-Solomon RS (15,9) encoder then generates the six parity symbols, represented by 12 additional probe pools. Thus, a total of 19 probe pools (7+12) are required to obtain error correction fort=3 symbols.

Monte Carlo simulations of error-correcting code performance have been performed assuming seven probe pools, to identify up to 16,384 distinct targets. Using these simulations, the maximum permissible raw error rate (associated with identifying a fluorescent label) to achieve a corrected error rate of 10-5 was determined for different numbers of parity bits.

In some embodiments, a key is generated that includes the expected bits of information associated with an analyte (e.g., the expected order of probes and types of signals for the analyte). These expected bits of information for a particular analyte are compared with the actual L bits of information that are obtained from the target analyte. Using the Reed-Solomon approach, an allowance of up tot errors in the signals can be tolerated in the comparison of the expected bits of information and the actual L bits of information.

In some embodiments, a Reed-Solomon decoder is used to compare the expected signal sequence with an observed signal sequence from a particular probe. For example, seven probe pools may be used to identify a target analyte, the expected color sequence being BGGBBYY, represented by 14 bits. Additional parity pools may then be used for error correction. For example, six 4-bit parity symbols may be used.

Methods and systems using cycled probe binding and optical detection are described in US Publication No. 2015/0330974, Digital Analysis of Molecular Analytes Using Single Molecule Detection, published Nov. 19, 2015, and US Publication No. 2018/0252936, High Speed Scanning With Acceleration Tracking, published Sep. 6, 2018, are each incorporated herein by reference herein in its entirety.

In some embodiments, the raw images are obtained using sampling that is at least at the Nyquist limit to facilitate more accurate determination of the oversampled image. Increasing the number of pixels used to represent the image by sampling in excess of the Nyquist limit (oversampling) increases the pixel data available for image processing and display.

Theoretically, a bandwidth-limited signal can be perfectly reconstructed if sampled at the Nyquist rate or above it. The Nyquist rate is defined as twice the highest frequency component in the signal. Oversampling improves resolution, reduces noise and helps avoid aliasing and phase distortion by relaxing anti-aliasing filter performance requirements. A signal is said to be oversampled by a factor of N if it is sampled at N times the Nyquist rate.

Thus, in some embodiments, each image is taken with a pixel size no more than half the wavelength of light being observed. In some embodiments, a pixel size of less than about 200 nm×200 nm is used in detection to achieve sampling at or above the Nyquist limit. Sampling at a frequency of at least the Nyquist limit during raw imaging of the substrate is preferred to optimize the resolution of the system or methods described herein. This can be done in conjunction with the deconvolution methods and optical systems described herein to resolve features on a substrate below the diffraction limit with high accuracy.

Processing Images from Different Cycles

There are several barriers overcome by the present invention to achieve sub-diffraction limited imaging.

Pixelation error is present in raw images and prevents identification of information present from the optical signals due to pixelation. Sampling at least at the Nyquist frequency and generation of an oversampled image as described herein each assist in overcoming pixilation error.

The point-spread (PSF) of various molecules overlap because the PSF size is greater than the pixel size (below Nyquist) and because the center-to-center spacing is so small that crosstalk due to spatial overlap occurs. Nearest neighbor e.g. variable regression (for center-to center crosstalk correction) can be used to help with deconvolution of multiple overlapping optical signals. But this can be improved if we know the relative location of each analyte on the substrate and have good alignment of images of a field. In some embodiments, machine learning (e.g. artificial intelligence or “A.I.”) can be used to help with deconvolution of multiple overlapping optical signals. In some embodiments, the machine learning processes input data over multiple cycles of probe binding and imaging to deconvolve further images.

After multiple cycles the precise location of the molecule may become increasingly more accurate. Using this information additional calculations can be performed to aid in deconvolution by correcting for known asymmetries in the spatial overlap of optical signals occurring due to pixel discretization effects and the diffraction limit. They can also be used to correct for overlap in emission spectrum from different emission spectrum.

Highly accurate relative positional information for each analyte can be achieved by overlaying images of the same field from different cycles to generate a distribution of measured peaks from optical signals of different probes bound to each analyte. This distribution can then be used to generate a peak signal that corresponds to a single relative location of the analyte. Images from a subset of cycles can be used to generate relative location information for each analyte. In some embodiments, this relative position information is provided in a localization file.

The specific area imaged for a field for each cycle may vary from cycle to cycle. Thus, to improve the accuracy of identification of analyte position for each image, an alignment between images of a field across multiple cycles can be performed. From this alignment, offset information compared to a reference file can then be identified and incorporated into the deconvolution algorithms to further increase the accuracy of deconvolution and signal identification for optical signals obscured due to the diffraction limit. In some embodiments, this information is provided in a Field Alignment File.

### Signal Detection (Cross-Talk/Nearest Neighbor)

Once relative positional information is accurately determined for analytes on a substrate and field images from each cycle are aligned with this positional information, analysis of each oversampled image using crosstalk and nearest neighbor regression can be used to accurately identify an optical signal from each analyte in each image.

In some embodiments, a plurality of optical signals obscured by the diffraction limit of the optical system are identified for each of a plurality of biomolecules deposited on a substrate and bound to probes comprising a detectable label. In some embodiments, the probes are incorporated nucleotides and the series of cycles is used to determine a sequence of a polynucleotide deposited on the array using sequencing by synthesis.

### Simulations of Deconvolution Applied to Images

Molecular Densities are Limited by Crosstalk from Neighboring Molecules

The physical size of the molecule may broaden the spot roughly half the size of the binding area. For example, for an 80 nm spot the pitch may be increased by roughly 40 nm. Smaller spot sizes may be used, but this may have the trade-off that fewer copies may be allowed and greater illumination intensity may be required. A single copy provides the simplest sample preparation but requires the greatest illumination intensity.

Methods for sub-diffraction limit imaging discussed to this point involve image processing techniques of oversampling, deconvolution and crosstalk correction. Described herein are methods and systems that incorporate determination of the precise relative location analytes on the substrate using information from multiple cycles of probe optical signal imaging for the analytes. Using this information additional calculations can be performed to aid in crosstalk correction regarding known asymmetries in the crosstalk matrix occurring due to pixel discretization effects.

### Methods

In some embodiments, provided herein is a method for accurately determining a relative position of analytes deposited on the surface of a densely packed substrate. The method includes first providing a substrate comprising a surface, wherein the surface comprises a plurality of analytes deposited on the surface at discrete locations. Then, a plurality of cycles of probe binding and signal detection on said surface is performed. Each cycle of detection includes contacting the analytes with a probe set capable of binding to target analytes deposited on the surface, imaging a field of said surface with an optical system to detect a plurality of optical signals from individual probes bound to said analytes at discrete locations on said surface, and removing bound probes if another cycle of detection is to be performed. From each image, a peak location from each of said plurality of optical signals from images of said field from at least two (i.e., a subset) of said plurality of cycles is detected. The location of peaks for each analyte is overlaid, generating a cluster of peaks from which an accurate relative location of each analyte on the substrate is then determined.

In some embodiments the accurate position information for analytes on the substrate is then used in a deconvolution algorithm incorporating position information (e.g., for identifying center-to-center spacing between neighboring analytes on the substrate) can be applied to the image to deconvolve overlapping optical signals from each of said images. In some embodiments, the deconvolution algorithm includes nearest neighbor variable regression for spatial discrimination between neighboring analytes with overlapping optical signals.

In some embodiments the method of analyte detection is applied for sequencing of individual polynucleotides deposited on a substrate.

In some embodiments, optical signals are deconvolved from densely packed substrates. The operations can be divided into four different sections. Image Analysis, which includes generation of oversampled images from each image of a field for each cycle, and generation of a peak file (i.e., a data set) including peak location and intensity for each detected optical signal in an image. 2) Generation of a Localization File, which includes alignment of multiple peaks generated from the multiple cycles of optical signal detection for each analyte to determining an accurate relative location of the analyte on the substrate. 3) Generation of a Field Alignment file, which includes offset information for each image to align images of the field from different cycles of detection with respect to a selected reference image. 4) Extract Intensities, which uses the offset information and location information in conjunction with deconvolution modeling to determine an accurate identity of signals detected from each oversampled image. The “Extract Intensities” operation can also include other error correction, such as previous cycle regression used to correct for errors in sequencing by synthesis processing and detection. The operations performed in each section are described in further detail below.

Under the image analysis operations, the images of each field from each cycle are processed to increase the number of pixels for each detected signal, sharpen the peaks for each signal, and identify peak intensities form each signal. This information is used to generate a peak file for each field for each cycle that includes a measure of the position of each analyte (from the peak of the observed optical signal), and the intensity, from the peak intensity from each signal. In some embodiments, the image from each field first undergoes background subtraction to perform an initial removal of noise from the image. Then, the images are processed using smoothing and deconvolution to generate an oversampled image, which includes artificially generated pixels based on modeling of the signal observed in each image. In some embodiments, the oversampled image can generate 4 pixels, 9 pixels, or 16 pixels from each pixel from the raw image.

Peaks from optical signals detected in each raw image or present in the oversampled image are then identified and intensity and position information for each detected analyte is placed into a peak file for further processing.

In some embodiments, N raw images corresponding to all images detected from each cycle and each field of a substrate or output into N oversampled images and N peak files for each imaged field. The peak file comprises a relative position of each detected analyte for each image. In some embodiments, the peak file also comprises intensity information for each detected analyte. In some embodiments, one peak file is generated for each color and each field in each cycle. In some embodiments, each cycle further comprises multiple passes, such that one peak file can be generated for each color and each field for each pass in each cycle. In some embodiments, the peak file specifies peak locations from optical signals within a single field.

In preferred embodiments, the peak file includes XY position information from each processed oversampled image of a field for each cycle. The XY position information comprises estimated coordinates of the locations of each detected detectable label from a probe (such as a fluorophore) from the oversampled image. The peak file can also include intensity information from the signal from each individual detectable label.

Generation of an oversampled image is used to overcome pixelation error to identify information present that cannot be extracted due to pixelation. Initial processing of the raw image by smoothing and deconvolution helps to provide more accurate information in the peak files so that the position of each analyte can be determined with higher accuracy, and this information subsequently can be used to provide a more accurate determination of signals obscured in diffraction limited imaging.

In some embodiments, the raw images are obtained using sampling that is at least at the Nyquist limit to facilitate more accurate determination of the oversampled image. Increasing the number of pixels used to represent the image by sampling in excess of the Nyquist limit (oversampling) increases the pixel data available for image processing and display.

Theoretically, a bandwidth-limited signal can be perfectly reconstructed if sampled at the Nyquist rate or above it. The Nyquist rate is defined as twice the highest frequency component in the signal. Oversampling improves resolution, reduces noise and helps avoid aliasing and phase distortion by relaxing anti-aliasing filter performance requirements. A signal is said to be oversampled by a factor of N if it is sampled at N times the Nyquist rate.

Thus, in some embodiments, each image is taken with a pixel size no more than half the wavelength of light being observed. In some embodiments, a pixel size of less than about 200 nm×200 nm is used in detection to achieve sampling at or above the Nyquist limit.

Smoothing uses an approximating function capture important patterns in the data, while leaving out noise or other fine-scale structures/rapid phenomena. In smoothing, the data points of a signal are modified so individual points are reduced, and points that are lower than the adjacent points are increased leading to a smoother signal. Smoothing is used herein to smooth the diffraction limited optical signal detected in each image to better identify peaks and intensities from the signal.

Although each raw image is diffraction limited, described herein are methods that result in collection of multiple signals from the same analyte from different cycles. These multiple signals from each analyte are used to determine a position much more accurate than the diffraction limited signal from each individual image. They can be used to identify molecules within a field at a resolution of less than 5 nm. This information is then stored as a localization file. The highly accurate position information can then be used to greatly improve signal identification from each individual field image in combination with deconvolution algorithms, such as cross-talk regression and nearest neighbor variable regression.

The operations for generating a localization file use the location information provided in the peak files to determine relative positions of a set of analytes on the substrate. In some embodiments, each localization file contains relative positions from sets of analytes from a single imaged field of the substrate. The localization file combines position information from multiple cycles to generate highly accurate position information for detected analytes below the diffraction limit.

In some embodiments, the relative position information for each analyte is determined on average to less than a 10 nm standard deviation (i.e., RMS, or root mean square). In some embodiments, the relative position information for each analyte is determined on average to less than a 10 nm 2× standard deviation. In some embodiments, the relative position information for each analyte is determined on average to less than a 10 nm 3× standard deviation. In some embodiments, the relative position information for each analyte is determined to less than a 10 nm median standard deviation. In some embodiments, the relative position information for each analyte is determined to less than a 10 nm median 2× standard deviation. In some embodiments, the relative position information for each analyte is determined to less than a 10 nm median 3× standard deviation.

From a subset of peak files for a field from different cycles, a localization file is generated to determine a location of analytes on the array. In some embodiments, a peak file is first normalized using a point spread function to account for aberrations in the optical system. The normalized peak file can be used to generate an artificial normalized image based on the location and intensity information provided in the peak file. Each image is then aligned. In some embodiments, the alignment can be performed by correlating each image pair and performing a fine fit. Once aligned, position information for each analyte from each cycle can then be overlaid to provide a distribution of position measurements on the substrate. This distribution is used to determine a single peak position that provides a highly accurate relative position of the analyte on the substrate. In some embodiments, a Poisson distribution is applied to the overlaid positions for each analyte to determine a single peak.

The peaks determined from at least a subset of position information from the cycles are then recorded in a localization file, which comprises a measure of the relative position of each detected analyte with an accuracy below the diffraction limit. As described, images from only subset of cycles are needed to determine this information.

A normalized peak file from each field for each cycle and color and the normalized localization file can be used to generate offset information for each image from a field relative to a reference image of the field. This offset information can be used to improve the accuracy of the relative position determination of the analyte in each raw image for further improvements in signal identification from a densely packed substrate and a diffraction limited image. In some embodiments, this offset information is stored as a field alignment file. In some embodiments, the position information of each analyte in a field from the combined localization file and field alignment file is less than 10 nm RMS, less than 5 nm RMS, or less than 2 nm RMS.

In some embodiments, a field alignment file is generated by alignment of images from a single field by determining offset information relative to a master file from the field. One field alignment file is generated for each field. This file is generated from all images of the field from all cycles, and includes offset information for all images of the field relative to a reference image from the field.

In some embodiments, before alignment, each peak file is normalized with a point spread function, followed by generation of an artificial image from the normalized peak file and Fourier transform of the artificial image. The Fourier transform of the artificial image of the normalized peak file is then convolved with a complex conjugate of the Fourier transform of an artificial image from the normalized localization file for the corresponding field. This is done for each peak file for each cycle. The resulting files then undergo an inverse Fourier transform to regenerate image files, and the image files are aligned relative to the reference file from the field to generate offset information for each image file. In some embodiments, this alignment includes a fine fit relative to a reference file.

The field alignment file thus contains offset information for each oversampled image, and can be used in conjunction with the localization file for the corresponding field to generate highly accurate relative position for each analyte for use in the subsequent “Extract Intensities” operations.

As an example where 20 cycles are performed on a field, and one image is generated for each of 4 colors to be detected, thus generating 80 images of the field, one Field Alignment file is generated for all 80 images (20 cycles*4 colors) taken of the field. In some embodiments, the field alignment file contents include: the field, the color observed for each image, the operation type in the cycled detection (e.g., binding or stripping), and the image offset coordinates relative to the reference image.

In some embodiments, during the alignment process XY “shifts” or “residuals” needed to align 2 images are calculated, and the process is repeated for remaining images, best fit residual to apply to all is calculated.

In some embodiments, residuals that exceed a threshold are thrown out, and best fit is re-calculated. This process is repeated until all individual residuals are within the threshold

Each oversampled image is then deconvolved using the accurate position information from the localization file and the offset information from the field alignment file. The Point Spread Function (PSF) of various molecules overlap because the center-to-center spacing is so small that the point-spread function of signals from adjacent analytes overlaps. Nearest neighbor variable regression in combination with the accurate analyte position information and/or offset information can be used to deconvolve signals from adjacent analytes that have a center-to-center distance that inhibits resolution due to the diffraction limit. The use of the accurate relative position information for each analyte facilitates spatial deconvolution of optical signals from neighboring analytes below the diffraction limit. In some embodiments, the relative position of neighboring analytes is used to determine an accurate center-to-center distance between neighboring analytes, which can be used in combination with the point spread function of the optical system to estimate spatial cross-talk between neighboring analytes for use in deconvolution of the signal from each individual image. This enables the use of substrates with a density of analytes below the diffraction limit for optical detection techniques, such as polynucleotide sequencing.

In certain embodiments, emission spectra overlap between different signals (i.e. “cross-talk”). For example, during sequencing by synthesis, the four dyes used in the sequencing process typically have some overlap in emission spectra.

In particular embodiments, a problem of assigning a color (for example, a base call) to different features in a set of images obtained for a cycle when crosstalk occurs between different color channels and when the crosstalk is different for different sets of images can be solved by cross-talk regression in combination with the localization and field alignment files for each oversampled image to remove overlapping emission spectrums from optical signals from each different detectable label used. This further increases the accuracy of identification of the detectable label identity for each probe bound to each analyte on the substrate.

Thus, in some embodiments, identification of a signal and/or its intensity from a single image of a field from a cycle as disclosed herein uses the following features: 1) Oversampled Image—provides intensities and signals at defined locations. 2) Accurate Relative Location—Localization File (provides location information from information from at least a subset of cycles) and Field Alignment File (provides offset/alignment information for all images in a field). 3) Image Processing—Nearest Neighbor Variable Regression (spatial deconvolution) and Cross-talk regression (emission spectra deconvolution) using accurate relative position information for each analyte in a field. Accurate identification of probes (e.g., antibodies for detection or complementary nucleotides for sequencing) for each analyte.

### Image Processing Simulations

The effects of the methods and systems disclosed herein are illustrated in simulated cross-talk plots. A cross-talk plot showing the intensity of emission spectrum correlated with one of four fluorophores at each detected analyte in a 10 um×10 um region is shown. Each axis corresponding to one of the four fluorophores extends to each corner of the plot. Thus, a spot located in the center of the plot may have equal contribution of intensity from all four fluorophores. Emission intensity detected from an individual fluorophore during an imaging cycle is assigned to move the spot in a direction either towards X, Y; X, −Y; −X, Y; or −X, ¬Y. Thus, separation of populations of spots along these four axes indicates a clear deconvolved signal from a fluorophore at an analyte location. Each simulation is based on detection of 1024 molecules in a 10.075 um×10.075 um region, indicating a density of 10.088 molecules per micron squared, or an average center-to-center distance between molecules of about 315 nm. This is correlated with an imaging region of about 62×62 pixels at a pixel size of less than about 200 nm×200 nm.

In some embodiments, the average center-to-center distance between molecules is about 150 nm to about 500 nm. In some embodiments, the average center-to-center distance between molecules is about 150 nm to about 175 nm, about 150 nm to about 200 nm, about 150 nm to about 225 nm, about 150 nm to about 250 nm, about 150 nm to about 275 nm, about 150 nm to about 300 nm, about 150 nm to about 325 nm, about 150 nm to about 350 nm, about 150 nm to about 375 nm, about 150 nm to about 400 nm, about 150 nm to about 500 nm, about 175 nm to about 200 nm, about 175 nm to about 225 nm, about 175 nm to about 250 nm, about 175 nm to about 275 nm, about 175 nm to about 300 nm, about 175 nm to about 325 nm, about 175 nm to about 350 nm, about 175 nm to about 375 nm, about 175 nm to about 400 nm, about 175 nm to about 500 nm, about 200 nm to about 225 nm, about 200 nm to about 250 nm, about 200 nm to about 275 nm, about 200 nm to about 300 nm, about 200 nm to about 325 nm, about 200 nm to about 350 nm, about 200 nm to about 375 nm, about 200 nm to about 400 nm, about 200 nm to about 500 nm, about 225 nm to about 250 nm, about 225 nm to about 275 nm, about 225 nm to about 300 nm, about 225 nm to about 325 nm, about 225 nm to about 350 nm, about 225 nm to about 375 nm, about 225 nm to about 400 nm, about 225 nm to about 500 nm, about 250 nm to about 275 nm, about 250 nm to about 300 nm, about 250 nm to about 325 nm, about 250 nm to about 350 nm, about 250 nm to about 375 nm, about 250 nm to about 400 nm, about 250 nm to about 500 nm, about 275 nm to about 300 nm, about 275 nm to about 325 nm, about 275 nm to about 350 nm, about 275 nm to about 375 nm, about 275 nm to about 400 nm, about 275 nm to about 500 nm, about 300 nm to about 325 nm, about 300 nm to about 350 nm, about 300 nm to about 375 nm, about 300 nm to about 400 nm, about 300 nm to about 500 nm, about 325 nm to about 350 nm, about 325 nm to about 375 nm, about 325 nm to about 400 nm, about 325 nm to about 500 nm, about 350 nm to about 375 nm, about 350 nm to about 400 nm, about 350 nm to about 500 nm, about 375 nm to about 400 nm, about 375 nm to about 500 nm, or about 400 nm to about 500 nm. In some embodiments, the average center-to-center distance between molecules is about 150 nm, about 175 nm, about 200 nm, about 225 nm, about 250 nm, about 275 nm, about 300 nm, about 325 nm, about 350 nm, about 375 nm, about 400 nm, or about 500 nm. In some embodiments, the average center-to-center distance between molecules is at least about 150 nm, about 175 nm, about 200 nm, about 225 nm, about 250 nm, about 275 nm, about 300 nm, about 325 nm, about 350 nm, about 375 nm, or about 400 nm. In some embodiments, the average center-to-center distance between molecules is at most about 175 nm, about 200 nm, about 225 nm, about 250 nm, about 275 nm, about 300 nm, about 325 nm, about 350 nm, about 375 nm, about 400 nm, or about 500 nm.

### Sequencing

The methods described above also facilitate sequencing by sequencing by synthesis using optical detection of complementary reversible terminators incorporated into a growing complementary strand on a substrate comprising densely packed polynucleotides. Thus, signals correlating with the sequence of neighboring polynucleotides at a center-to-center distance below the diffraction limit can be reliably detected using the methods and optical detection systems described herein. Image processing during sequencing can also include previous cycle regression based on clonal sequences repeated on the substrate or on the basis of the data itself to correct for errors in the sequencing reaction or detection. In some embodiments, the polynucleotides deposited on the substrate for sequencing are concatemers. A concatemer can comprise multiple identical copies of a polynucleotide to be sequenced. Thus, each optical signal identified by the methods and systems described herein can refer to a single detectable label (e.g., a fluorophore) from an incorporated nucleotide, or can refer to multiple detectable labels bound to multiple locations on a single concatemer, such that the signal is an average from multiple locations. The resolution that may occur may not be between individual detectable labels, but between different concatemers deposited to the substrate.

In some embodiments, molecules to be sequenced, single or multiple copies, may be bound to the surface using covalent linkages, by hybridizing to capture oligonucleotide on the surface, or by other non-covalent binding. The bound molecules may remain on the surface for hundreds of cycles and can be re-interrogated with different primer sets, following stripping of the initial sequencing primers, to confirm the presence of specific variants.

In one embodiment, the fluorophores and blocking groups may be removed using chemical reactions.

In another embodiment, the fluorescent and blocking groups may be removed using UV light.

In one embodiment, the molecules to be sequenced may be deposited on reactive surfaces that have 50-100 nM diameters and these areas may be spaced at a pitch of 150-300 nM. These molecules may have barcodes, attached onto them for target de-convolution and a sequencing primer binding region for initiating sequencing. Buffers may contain appropriate amounts of DNA polymerase to enable an extension reaction. These may contain 10-100 copies of the target to be sequenced generated by any of the gene amplification methods available (PCR, whole genome amplification etc.)

In another embodiment, single target molecules, tagged with a barcode and a primer annealing site may be deposited on a 20-50 nM diameter reactive surface spaced with a pitch of 60-150 nM. The molecules may be sequenced individually.

In one embodiment, a primer may bind to the target and may be extended using one dNTP at a time with a single or multiple fluorophore (s); the surface may be imaged, the fluorophore may be removed and washed and the process repeated to generate a second extension. The presence of multiple fluorophores on the same dNTP may enable defining the number of repeats nucleotides present in some regions of the genome (2 to 5 or more).

In a different embodiment, following primer annealing, all four dNTPs with fluorophores and blocked 3′ hydroxyl groups may be used in the polymerase extension reaction, the surface may be imaged and the fluorophore and blocking groups removed and the process repeated for multiple cycles.

In another embodiment, the sequences may be inferred based on ligation reactions that anneal specific probes that ligate based on the presence of a specific nucleotides at a given position.

A random array may be used which may have improved densities over prior art random arrays using the techniques outlined above, however random arrays generally have 4× to 10× reduced areal densities of ordered arrays. Advantages of a random array include a uniform, non-patterned surface for the chip and the use of shorter nucleic acid strands because there is no need to rely on the exclusionary properties of longer strands.

### Computer Systems

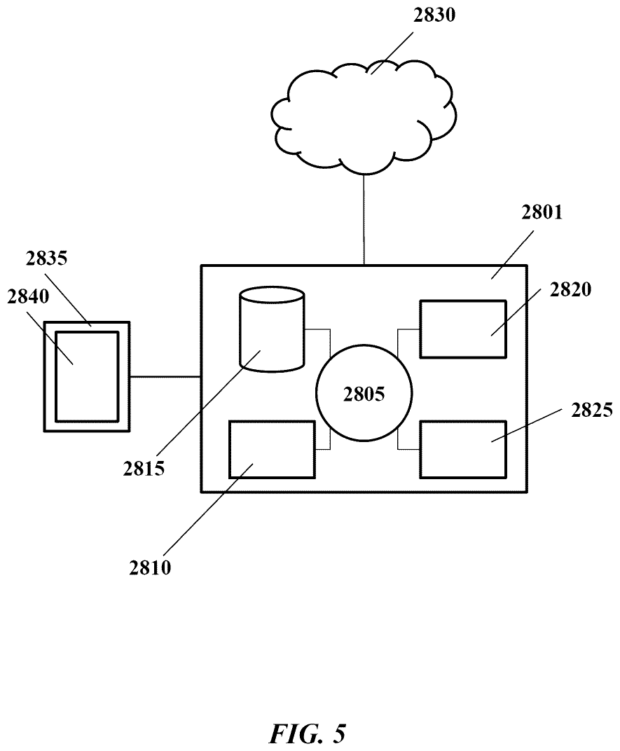

The present disclosure provides computer systems that are programmed to implement methods of the disclosure. An example is depicted in FIG. 5. The computer system 2801 that is programmed or otherwise configured to direct the methods described herein and utilize the systems described herein. The computer system 2801 can regulate various aspects of the present disclosure, such as, for example, directing the cycles of probe binding described herein. The computer system 2801 can be an electronic device of a user or a computer system that is remotely located with respect to the electronic device. The electronic device can be a mobile electronic device.

The computer system 2801 includes a central processing unit (CPU, also “processor” and “computer processor” herein) 2805, which can be a single core or multi core processor, or a plurality of processors for parallel processing. The computer system 2801 also includes memory or memory location 2810 (e.g., random-access memory, read-only memory, flash memory), electronic storage unit 2815 (e.g., hard disk), communication interface 2820 (e.g., network adapter) for communicating with one or more other systems, and peripheral devices 2825, such as cache, other memory, data storage and/or electronic display adapters. The memory 2810, storage unit 2815, interface 2820 and peripheral devices 2825 are in communication with the CPU 2805 through a communication bus (solid lines), such as a motherboard. The storage unit 2815 can be a data storage unit (or data repository) for storing data. The computer system 2801 can be operatively coupled to a computer network (“network”) 2830 with the aid of the communication interface 2820. The network 2830 can be the Internet, an internet and/or extranet, or an intranet and/or extranet that is in communication with the Internet. The network 2830 in some cases is a telecommunication and/or data network. The network 2830 can include one or more computer servers, which can enable distributed computing, such as cloud computing. The network 2830, in some cases with the aid of the computer system 2801, can implement a peer-to-peer network, which may enable devices coupled to the computer system 2801 to behave as a client or a server.

The CPU 2805 can execute a sequence of machine-readable instructions, which can be embodied in a program or software. The instructions may be stored in a memory location, such as the memory 2810. The instructions can be directed to the CPU 2805, which can subsequently program or otherwise configure the CPU 2805 to implement methods of the present disclosure. Examples of operations performed by the CPU 2805 can include fetch, decode, execute, and writeback.

The CPU 2805 can be part of a circuit, such as an integrated circuit. One or more other components of the system 2801 can be included in the circuit. In some cases, the circuit is an application specific integrated circuit (ASIC).

The storage unit 2815 can store files, such as drivers, libraries and saved programs. The storage unit 2815 can store user data, e.g., user preferences and user programs. The computer system 2801 in some cases can include one or more additional data storage units that are external to the computer system 2801, such as located on a remote server that is in communication with the computer system 2801 through an intranet or the Internet.

The computer system 2801 can communicate with one or more remote computer systems through the network 2830. For instance, the computer system 2801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2801 via the network 2830.

Methods as described herein can be implemented by way of machine (e.g., computer processor) executable code stored on an electronic storage location of the computer system 2801, such as, for example, on the memory 2810 or electronic storage unit 2815. The machine executable or machine readable code can be provided in the form of software. During use, the code can be executed by the processor 2805. In some cases, the code can be retrieved from the storage unit 2815 and stored on the memory 2810 for ready access by the processor 2805. In some situations, the electronic storage unit 2815 can be precluded, and machine-executable instructions are stored on memory 2810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 2801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2801 can include or be in communication with an electronic display 2835 that comprises a user interface (UI) 2840 for providing, for example, the detectable signal sequences mentioned herein or the identification of analytes as mentioned herein or the location of analytes as disclosed herein or any other information disclosed herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2805. The algorithm can, for example, direct the optical modules disclosed herein to capture an image or direct probe binding.

### Definitions

Those skilled in the art will recognize, or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “center-to-center distance” generally refers to a distance between two adjacent molecules as measured by the difference between the average position of each molecule on a substrate. The term average minimum center-to-center distance refers specifically to the average distance between the center of each analyte disposed on the substrate and the center of its nearest neighboring analyte, although the term center-to-center distance refers also to the minimum center-to-center distance in the context of limitations corresponding to the density of analytes on the substrate. As used herein, the term “pitch” or “average effective pitch” is generally used to refer to average minimum center-to-center distance. In the context of regular arrays of analytes, pitch may also be used to determine a center-to-center distance between adjacent molecules along a defined axis.

As used herein, the term “overlaying” (e.g., overlaying images) generally refers to overlaying images from different cycles to generate a distribution of detected optical signals (e.g., position and intensity, or position of peak) from each analyte over a plurality of cycles. This distribution of detected optical signals can be generated by overlaying images, overlaying artificial processed images, or overlaying datasets comprising positional information. Thus, as used herein, the term “overlaying images” generally encompasses any of these mechanisms to generate a distribution of position information for optical signals from a single probe bound to a single analyte for each of a plurality of cycles.

A “cycle” is generally defined by completion of one or more passes and stripping of the detectable label from the substrate. Subsequent cycles of one or more passes per cycle can be performed. For the methods and systems described herein, multiple cycles are performed on a single substrate or sample. For deoxyribonucleic acid (DNA) sequencing, multiple cycles may require the use of a reversible terminator and a removable detectable label from an incorporated nucleotide. For proteins, multiple cycles may require that the probe removal (stripping) conditions either maintain proteins folded in their proper configuration, or that the probes used are chosen to bind to peptide sequences so that the binding efficiency is independent of the protein fold configuration.

A “pass” in a detection assay generally refers to a process where a plurality of probes comprising a detectable label are introduced to the bound analytes, selective binding occurs between the probes and distinct target analytes, and a plurality of signals are detected from the detectable labels. A pass includes introduction of a set of antibodies that bind specifically to a target analyte. A pass can also include introduction of a set of labelled nucleotides for incorporation into the growing strand during sequencing by synthesis. There can be multiple passes of different sets of probes before the substrate is stripped of all detectable labels, or before the detectable label or reversible terminator is removed from an incorporated nucleotide during sequencing. In general, if four nucleotides are used during a pass, a cycle may only include a single pass for standard four nucleotide sequencing by synthesis.

As used herein, an “image” generally refers to an image of a field taken during a cycle or a pass within a cycle. In some embodiments, a single image is limited to detection of a single color of a detectable label.

As used herein, the term “field” generally refers to a single region of a substrate that is imaged. During a typical assay a single field is imaged at least once per cycle. For example, for a 20 cycle assay, with 4 colors, there can be 20*4=80 images, all of the same field.

A “target analyte” or “analyte” generally refers to a molecule, compound, complex, substance or component that is to be identified, quantified, and otherwise characterized. A target analyte can comprise by way of example, but not limitation to, a single molecule (of any molecular size), a single biomolecule, a polypeptide, a protein (folded or unfolded), a polynucleotide molecule (ribonucleic acid (RNA), complementary DNA (cDNA), or DNA), a fragment thereof, a modified molecule thereof, such as a modified nucleic acid, or a combination thereof. In an embodiment, a target polynucleotide comprises a hybridized primer to facilitate sequencing by synthesis. The target analytes are recognized by probes, which can be used to sequence, identify, and quantify the target analytes using optical detection methods described herein.

A “probe,” as used herein generally refers to a molecule that is capable of binding to other molecules (e.g., a complementary labelled nucleotide during sequencing by synthesis, polynucleotides, polypeptides or full-length proteins, etc.), cellular components or structures (lipids, cell walls, etc.), or cells for detecting or assessing the properties of the molecules, cellular components or structures, or cells. The probe comprises a structure or component that binds to the target analyte. In some embodiments, multiple probes may recognize different parts of the same target analyte. Examples of probes include, but are not limited to, a labelled reversible terminator nucleotide, an aptamer, an antibody, a polypeptide, an oligonucleotide (DNA, RNA), or any combination thereof. Antibodies, aptamers, oligonucleotide sequences and combinations thereof as probes are also described in detail below.

The probe can comprise a detectable label that is used to detect the binding of the probe to a target analyte. The probe can be directly or indirectly bound to, hybridized to, conjugated to, or covalently linked to the target analyte.

As used herein, the term “detectable label” generally refers to a molecule bound to a probe that can generate a detectable optical signal when the probe is bound to a target analyte and imaged using an optical imaging system. The detectable label can be directly or indirectly bound to, hybridized to, conjugated to, or covalently linked to the probe. In some embodiments, the detectable label is a fluorescent molecule or a chemiluminescent molecule. The probe can be detected optically via the detectable label.

As used herein, the term “optical distribution model” generally refers to a statistical distribution of probabilities for light detection from a point source. These include, for example, a Gaussian distribution. The Gaussian distribution can be modified to include anticipated aberrations in detection to generate a point spread function as an optical distribution model.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### Embodiments

Also described herein are the following embodiments:
1. A system comprising an analyte disposed adjacent to a substrate, wherein said analyte has a first dimension and a second dimension, wherein said first dimension is along an axis parallel to said substrate and said second dimension is along an axis orthogonal to said substrate, wherein said first dimension is less than a diffraction limit of an optical system (λ/(2*NA)) configured to image said analyte, and wherein said second dimension is less than one-half of a depth-of-focus of said optical system (λ/(2*NA\hat{ }2)).
2. The system of embodiment 1, wherein said analyte is a nucleic acid concatemer.
3. The system of embodiment 1, wherein said analyte is a protein.
4. The system of embodiment 1, wherein said analyte is deoxyribonucleic acid (DNA), ribonucleic acid (RNA), messenger ribonucleic acid (mRNA), or any combination thereof.
5. The system of embodiment 4, wherein said DNA or RNA is single stranded.
6. The system of embodiment 1, wherein said one or more analytes are bound to a support, wherein said support is immobilized on said substrate.
7. The system of embodiment 6, wherein said support is UV treated.
8. The system of embodiment 6, wherein said support is spherical or circular.
9. The system of embodiment 6, wherein said support is a nucleic acid origami structure.
10. The system of embodiment 9, wherein said nucleic acid origami structure comprises a nucleic acid molecule.
11. The system of embodiment 10, wherein said nucleic acid molecule is DNA or RNA.
12. The system of embodiment 11, wherein said DNA or RNA is single stranded.
13. The system of embodiment 6, wherein said support is a circular disk and said analyte is bound to a single side of said circular disk.
14. The system of embodiment 6, wherein said support is a metal or non-metal nanoball.
15. The system of embodiment 14, wherein said metal or non-metal nanoball comprises carbon.
16. The system of embodiment 6, wherein said support comprises linkers configured to bind to said analyte.
17. The system of embodiment 16, wherein said linkers are nucleic acid primers.
18. The system of embodiment 16, wherein said analyte comprises repeating regions.
19. The system of embodiment 18, wherein said linkers are configured to bind to said repeating regions of said analyte.
20. The system of embodiment 16, wherein said linkers comprise nucleic acid molecules.
21. The system of embodiment 20, wherein said nucleic acid molecules are DNA or RNA.
22. The system of embodiment 21, wherein said DNA or RNA is double stranded.
23. The system of embodiment 21, wherein said DNA or RNA is single stranded.
24. The system of embodiment 18, wherein said repeating regions comprise one or more known sequences.
25. The system of embodiment 24, wherein said one or more known sequences comprise a barcode sequence, an adaptor sequence, a primer sequence, or any combination thereof.
26. The system of embodiment 16, wherein a melting point of said linkers is greater than a temperature reached during processing of said analyte.
27. The system of embodiment 1, wherein said substrate comprises one or more artifacts adjacent to said analyte, wherein said one or more artifacts do not generate a signal.
28. The system of embodiment 27, wherein said one or more artifacts generate a neighboring effect on said analyte.
29. The system of embodiment 28, wherein said neighboring effect comprises immobilizing analyte.
30. The system of embodiment 27, wherein at least 10% of said one or more artifacts comprise a first dimension along the axis parallel to the substrate of less than the diffraction limit of said optical system configured to image said analyte when said one or more artifacts are deposited adjacent to said analyte, and wherein at least 10% of said one or more artifacts comprise a second dimension along the axis orthogonal to the substrate of less than one-half of the depth-of-focus of said optical system configured to image said analyte when said one or more artifacts are deposited adjacent to said analyte.
31. The system of embodiment 1, wherein said analyte comprises a scaffold.
32. The system of embodiment 31, wherein said scaffold comprises at least two scaffolds wherein said at least two scaffolds are double stranded and intersect at a point of intersection.
33. The system of embodiment 32, wherein said point of intersection is a Holliday junction.
34. The system of embodiment 32, wherein at said point of intersection said at least two scaffolds are bound together.
35. The system of embodiment 31, wherein said scaffold comprises one or more biopolymers.
36. The system of embodiment 35, wherein said one or more biopolymers comprise a carbon-based polymer.
37. The system of embodiment 36, wherein said one or more biopolymers comprise a polyether.
38. The system of embodiment 35, wherein said one or more biopolymers comprise a polypeptide.
39. The system of embodiment 35, wherein said one or more biopolymers are detergent molecules.
40. The system of embodiment 35, wherein said one or more biopolymers comprise a nucleic acid molecule.
41. The system of embodiment 40, wherein said nucleic acid molecule is DNA or RNA.
42. The system of embodiment 41, wherein said DNA or RNA is double stranded.
43. The system of embodiment 41, wherein said DNA or RNA is single stranded.
44. The system of embodiment 43, wherein said one or more biopolymers is single stranded DNA, and said scaffold comprises at least two of said one or more biopolymers.
45. The system of embodiment 42, wherein said two biopolymers are oriented in a same direction.
46. The system of embodiment 43, wherein said same direction is 5′ to 3′.
47. The system of embodiment 44, wherein the 5′ terminal bases of said two biopolymers are linked via a linker.
48. The system of embodiment 45, wherein said linker is a covalent linker.
49. The system of embodiment 45, wherein said linker is an additional biopolymer.
50. The system of embodiment 47, wherein said additional biopolymer is a polypeptide.
51. The system of embodiment 47, wherein said additional biopolymer is an additional nucleic acid molecule.
52. The system of embodiment 40, wherein said nucleic acid molecule is DNA.
53. The system of embodiment 35, wherein said biopolymer comprises 1 to 500 monomers.
54. The system of embodiment 35, wherein said biopolymer comprises 50 to 400 monomers.
55. The system of embodiment 35, wherein said biopolymer comprises 100 monomers.
56. The system of embodiment 35, wherein said biopolymer is double stranded DNA and comprises 100 base pairs.
57. The system of embodiment 31, wherein said scaffold is configured to bind to repeating regions of said analyte.
58. The system of embodiment 53, wherein said repeating regions comprise one or more known sequences.
59. The system of embodiment 54, wherein said one or more known sequences comprise a barcode sequence, an adaptor sequence, a primer sequence, or any combination thereof.
60. The system of embodiment 31, wherein a melting point of said scaffold is greater than a temperature reached during processing of said analytes.
61. A method for processing one or more analytes, the method comprising:
   (a) depositing said one or more analytes adjacent to a substrate, wherein at least 10% of said one or more analytes comprise a first dimension along the axis parallel to the substrate of less than the diffraction limit of an optical system configured to image said one or more analytes when said one or more analytes are deposited adjacent to said substrate, and wherein at least 10% of said one or more analytes comprise a second dimension along the axis orthogonal to the substrate of less than one-half of the depth-of-focus of said optical system configured to image said one or more analytes when said one or more analytes are deposited adjacent to said substrate.
   (b) contacting said one or more analytes with a plurality of probes over a plurality of cycles, wherein said plurality of probes generate a plurality of signals;
   (c) obtaining said plurality of optical signals from said plurality of probes over said plurality of cycles of said plurality of probes binding to said one or more analytes deposited adjacent to said substrate; and
   (d) processing at least one optical signal of said plurality of optical signals to identify said one or more analytes of said plurality of analytes.
62. The method of embodiment 57, wherein said one or more analytes are nucleic acid concatemers.
63. The method of embodiment 57, wherein said one or more analytes are proteins.
64. The method of embodiment 57, wherein said one or more analytes are DNA, RNA, mRNA, or any combination thereof.
65. The method of embodiment 60, wherein said DNA or RNA is single stranded.
66. The method of embodiment 57, wherein said one or more analytes are bound to one or more support structures, wherein said one or more support structures are immobilized on said substrate.
67. The method of embodiment 62, wherein said one or more support structures are UV treated.
68. The method of embodiment 62, wherein a single analyte of said one or more analytes is bound to a single support structure of said one or more support structures.
69. The method of embodiment 62, wherein a single analyte of said one or more analytes is bound to a plurality of support structures of said one or more support structures.
70. The method of embodiment 62, wherein said support structure is spherical or circular.
71. The method of embodiment 62, wherein said support structure is a nucleic acid origami structure.
72. The method of embodiment 67, wherein said nucleic acid origami structure comprises a nucleic acid molecule.
73. The method of embodiment 68, wherein said nucleic acid molecule is DNA or RNA.
74. The method of embodiment 69, wherein said DNA or RNA is single stranded.
75. The method of embodiment 62, wherein said support structure is a circular disk and the one or more analytes are bound to a single side of the circular disk.
76. The method of embodiment 62, wherein said support structure is a metal or non-metal nanoball.
77. The method of embodiment 72, wherein said metal or non-metal nanoball comprises carbon.
78. The method of embodiment 62, wherein said support structure comprises linkers configured to bind to said one or more analytes.
79. The method of embodiment 74, wherein said linkers are nucleic acid primers.
80. The method of embodiment 74, wherein said one or more analytes comprise repeating regions.
81. The method of embodiment 76, wherein said linkers are configured to bind to said repeating regions of said one or more analytes.
82. The method of embodiment 74, wherein said linkers comprise nucleic acid molecules.
83. The method of embodiment 78, wherein said nucleic acid molecules are DNA or RNA.
84. The method of embodiment 79, wherein said DNA or RNA is double stranded.
85. The method of embodiment 79, wherein said DNA or RNA is single stranded.
86. The method of embodiment 57, wherein said support structure comprises one or more biopolymers.
87. The method of embodiment 80, wherein said one or more biopolymers is single stranded DNA, and said support structure comprises at least two of said one or more biopolymers.
88. The method of embodiment 81, wherein said two biopolymers are oriented in a same direction.
89. The method of embodiment 82, wherein said same direction is 5′ to 3′. 90. The method of embodiment 83, wherein the 5′ terminal bases of said two biopolymers are linked via a linker.
91. The method of embodiment 84, wherein said linker is a covalent linker.
92. The method of embodiment 84, wherein said linker is an additional biopolymer 93. The method of embodiment 86, wherein said additional biopolymer is a polypeptide.
94. The method of embodiment 86, wherein said additional biopolymer is an additional nucleic acid molecule.
95. The method of embodiment 76, wherein said repeating regions comprise one or more known sequences.
96. The method of embodiment 89, wherein said one or more known sequences comprise a barcode sequence, an adaptor sequence, a primer sequence, or any combination thereof.
97. The method of embodiment 74, wherein a melting point of said linkers is greater than a temperature reached during processing of said one or more analytes.
98. The method of embodiment 57, wherein said substrate comprises one or more artifacts adjacent to said one or more analytes, wherein said one or more artifacts do not generate a signal.
99. The method of embodiment 92, wherein said one or more artifacts generate a neighboring effect on said one or more analytes.
100. The method of embodiment 93, wherein said neighboring effect comprises immobilizing said one or more analytes.
101. The method of embodiment 57, wherein said one or more analytes comprise a scaffold.
102. The method of embodiment 95, wherein said scaffold comprises at least two scaffolds wherein said at least two scaffolds are double stranded and intersect at a point of intersection.
103. The method of embodiment 96, wherein said point of intersection is a Holliday junction.
104. The method of embodiment 96, wherein at said point of intersection of said at least two scaffolds are bound together.
105. The method of embodiment 95, wherein said scaffold comprises one or more biopolymers.
106. The method of embodiment 97, wherein said one or more biopolymers comprise a carbon-based polymer.
107. The method of embodiment 98, wherein said one or more biopolymers comprise a polyether.
108. The method of embodiment 97, wherein said one or more biopolymers comprise a polypeptide.
109. The method of embodiment 97, wherein said one or more biopolymers are detergent molecules.
110. The method of embodiment 97, wherein said one or more biopolymers comprise a nucleic acid molecule.
111. The method of embodiment 110Error! Reference source not found., wherein said nucleic acid molecule is DNA or RNA.
112. The method of embodiment 111, wherein said DNA or RNA is double stranded.
113. The method of embodiment 111, wherein said DNA or RNA is single stranded.
114. The method of embodiment 111, wherein said nucleic acid molecule is DNA.
115. The method of embodiment 97, wherein said biopolymer comprises 1 to 500 monomers.
116. The method of embodiment 97, wherein said biopolymer comprises 50 to 400 monomers.
117. The method of embodiment 97, wherein said biopolymer comprises 100 monomers.
118. The method of embodiment 97, wherein said biopolymer is double stranded DNA and comprises 100 base pairs.
119. The method of embodiment 95, wherein said scaffold is configured to bind to repeating regions of said one or more analytes.
120. The method of embodiment 104, wherein said repeating regions comprise a one or more known sequences.
121. The method of embodiment 105, wherein said one or more known sequences comprise a barcode sequence, an adaptor sequence, a primer sequence, or any combination thereof.
122. The method of embodiment 95, wherein a melting point of said scaffold is greater than a temperature reached during processing of said one or more analytes.
123. The method of embodiment 57, wherein a surface of said substrate comprises one or more anchor moieties configured to bind to said one or more analytes.
124. The method of embodiment 108, wherein said one or more anchor moieties comprise an antibody.
125. The method of embodiment 108, wherein said one or more anchor moieties are nucleic acid primers.
126. The method of embodiment 108, wherein said one or more anchor moieties are configured to bind to repeating regions of said one or more analytes.
127. The method of embodiment 108, wherein said one or more anchor moieties comprise nucleic acid molecules.
128. The method of embodiment 112, wherein said nucleic acid molecule is DNA or RNA.
129. The method of embodiment 128, wherein said DNA or RNA is double stranded.
130. The method of embodiment 128, wherein said DNA or RNA is single stranded.
131. The method of embodiment 104, wherein said repeating regions comprise a one or more known sequences.
132. The method of embodiment 113, wherein said one or more known sequences comprise a barcode sequence, an adaptor sequence, a primer sequence, or any combination thereof.
133. The method of embodiment 57, wherein said surface comprises one or more reagents to immobilize to one or more anchor moieties.
134. The method of embodiment 115, wherein said one or more reagents comprises streptavidin, polyethylene glycol (PEG), biotin, or any combination thereof.
135. The method of embodiment 115, wherein a melting point of said one or more anchor moieties is greater than a temperature reached during processing of said one or more analytes.
136. The method of embodiment 57, wherein a density of said one or more analytes does not exceed about 25 analytes per square micrometer.
137. The method of embodiment 57, wherein said one or more analytes first dimension along the axis parallel to the substrate are less than 400 nm.
138. The method of embodiment 57, wherein said one or more analytes first dimension along the axis parallel to the substrate are less than 300 nm.
139. The method of embodiment 57, wherein said one or more analytes first dimension along the axis parallel to the substrate are 200 nm to 300 nm.
140. The method of embodiment 57, wherein said one or more analytes first dimension along the axis parallel to the substrate are 100 nm to 200 nm.
141. The method of embodiment 57, wherein said one or more analytes first dimension along the axis parallel to the substrate are less than 200 nm.
142. A system for processing one or more analytes, the system comprising:
   (a) a substrate;
   (b) an optical system configured to image said substrate;
   (c) one or more analytes comprising a first dimension along the axis parallel to the substrate and a second dimension along the axis orthogonal to the substrate, wherein said first dimension along the axis parallel to the substrate is less than the diffraction limit of said optical system configured to image said one or more analytes when said one or more analytes are deposited adjacent to said substrate, and wherein said second dimension along the axis orthogonal to the substrate is less than one-half of the depth-of-focus of said optical system configured to image said one or more analytes when said one or more analytes are deposited adjacent to said substrate; and
   (d) a plurality of probes configured to generate a plurality of signals when bound to said one or more analytes.
143. The system of embodiment 142, wherein said one or more analytes are nucleic acid concatemers.
144. The system of embodiment 142, wherein said one or more analytes are proteins.
145. The system of embodiment 143, wherein said one or more analytes are DNA, RNA, mRNA, or any combination thereof.
146. The system of embodiment 145, wherein said DNA or RNA is single stranded.
147. The system of embodiment 142, wherein said one or more analytes are bound to a support, wherein said support is immobilized on said substrate.
148. The system of embodiment 147, wherein said support is UV treated.
149. The system of embodiment 147, wherein said support is spherical or circular.
150. The system of embodiment 147, wherein said support is a nucleic acid origami structure.
151. The system of embodiment 150, wherein said nucleic acid origami structure comprises a nucleic acid molecule.
152. The system of embodiment 151, wherein said nucleic acid molecule is DNA or RNA.
153. The system of embodiment 152, wherein said DNA or RNA is single stranded.
154. The system of embodiment 142, wherein said support is a circular disk and said one or more analytes are bound to a single side of the circular disk.
155. The system of embodiment 142, wherein said support is a metal or non-metal nanoball.
156. The system of embodiment 155, wherein said metal or non-metal nanoball comprises carbon.
157. The system of embodiment 142, wherein said support comprises linkers configured to bind to said one or more analytes.
158. The system of embodiment 157, wherein said linkers are nucleic acid primers.
159. The system of embodiment 158, wherein said one or more analytes comprise repeating regions.
160. The system of embodiment 159, wherein said linkers are configured to bind to said repeating regions of said one or more analytes.
161. The system of embodiment 160, wherein said linkers comprise nucleic acid molecules.
162. The system of embodiment 161, wherein said nucleic acid molecules are DNA or RNA.
163. The system of embodiment 162, wherein said DNA or RNA is double stranded.
164. The system of embodiment 163, wherein said DNA or RNA is single stranded.
165. The system of embodiment 159, wherein said repeating regions comprise a one or more known sequences.
166. The system of embodiment 165, wherein said one or more known sequences comprise a barcode sequence, an adaptor sequence, a primer sequence, or any combination thereof.
167. The system of embodiment 157, wherein a melting point of said linkers is greater than a temperature reached during processing of said one or more analytes.
168. The system of embodiment 142, wherein said one or more analytes comprise a scaffold.
169. The system of embodiment 168, wherein said scaffold comprises at least two scaffolds wherein said at least two scaffolds are double stranded and intersect at a point of intersection.
170. The system of embodiment 169, wherein said point of intersection is a Holliday junction.
171. The system of embodiment 169, wherein at said point of intersection said at least two scaffolds are bound together.
172. The system of embodiment 168, wherein said scaffold comprises one or more biopolymers.
173. The system of embodiment 172, wherein said one or more biopolymers comprise a carbon-based polymer.
174. The system of embodiment 173, wherein said one or more biopolymers comprise a polyether.
175. The system of embodiment 173, wherein said one or more biopolymers comprise a polypeptide.
176. The system of embodiment 173, wherein said one or more biopolymers are detergent molecules.
177. The system of embodiment 173, wherein said one or more biopolymers comprise a nucleic acid molecule.
178. The system of embodiment 177, wherein said nucleic acid molecule is DNA or RNA.
179. The system of embodiment 178, wherein said DNA or RNA is double stranded.
180. The system of embodiment 178, wherein said DNA or RNA is single stranded.
181. The system of embodiment 178, wherein said one or more biopolymers is single stranded DNA, and said scaffold comprises at least two of said one or more biopolymers.
182. The system of embodiment 181, wherein said two biopolymers are oriented in a same direction.
183. The system of embodiment 182, wherein said same direction is 5′ to 3′. 184. The system of embodiment 183, wherein the 5′ terminal bases of said two biopolymers are linked via a linker.
185. The system of embodiment 184, wherein said linker is a covalent linker.
186. The system of embodiment 185, wherein said linker is an additional biopolymer 187. The system of embodiment 186, wherein said additional biopolymer is a polypeptide.
188. The system of embodiment 186, wherein said additional biopolymer is an additional nucleic acid molecule.
189. The system of embodiment 188, wherein said nucleic acid molecule is DNA.
190. The system of embodiment 181, wherein said one or more biopolymer comprises 1 to 500 monomers.
191. The system of embodiment 181, wherein said one or more biopolymer comprises 50 to 400 monomers.
192. The system of embodiment 181, wherein said one or more biopolymer comprises 100 monomers.
193. The system of embodiment 181, wherein said one or more biopolymer is double stranded DNA and comprises 100 base pairs.
194. The system of embodiment 168, wherein said scaffold is configured to bind to said repeating regions of said one or more analytes.
195. The system of embodiment 194, wherein said repeating regions comprise one or more known sequences.
196. The system of embodiment 195, wherein said one or more known sequences comprise a barcode sequence, an adaptor sequence, a primer sequence, or any combination thereof.
197. The system of embodiment 168, wherein a melting point of said scaffold is greater than a temperature reached during processing of said one or more analytes.
198. The system of embodiment 142, wherein a surface of said substrate comprises one or more anchor moieties configured to bind to said one or more analytes.
199. The system of embodiment 198, wherein said one or more anchor moieties comprise an antibody.
200. The system of embodiment 198, wherein said one or more anchor moieties are nucleic acid primers.
201. The system of embodiment 198, wherein said one or more anchor moieties are configured to bind to said repeating regions of said one or more analytes.
202. The system of embodiment 198, wherein said one or more anchor moieties comprise nucleic acid molecules.
203. The system of embodiment 202, wherein said nucleic acid molecule is DNA or RNA.
204. The system of embodiment 203, wherein said DNA or RNA is double stranded.
205. The system of embodiment 203, wherein said DNA or RNA is single stranded.
206. The system of embodiment 201, wherein said repeating regions comprise a one or more known sequences.
207. The system of embodiment 206, wherein said one or more known sequences comprise a barcode sequence, an adaptor sequence, a primer sequence, or any combination thereof.
208. The system of embodiment 142, wherein said substrate comprises a surface, wherein said surface comprises one or more reagents to immobilize said one or more anchor moieties.
209. The system of embodiment 208, wherein said one or more reagents comprises streptavidin, polyethylene glycol (PEG), biotin, or any combination thereof.
210. The system of embodiment 198, wherein a melting point of said anchor moieties is greater than a temperature reached during processing of said one or more analytes.
211. The system of embodiment 142, wherein a density of said one or more analytes does not exceed about 25 analytes per square micrometer.
212. The system of embodiment 142, wherein said one or more analytes first dimension along the axis parallel to the substrate are less than 400 nm.
213. The system of embodiment 142, wherein said one or more analytes first dimension along the axis parallel to the substrate are less than 300 nm.
214. The system of embodiment 142, wherein said one or more analytes first dimension along the axis parallel to the substrate are 200 nm to 300 nm.
215. The system of embodiment 142, wherein said one or more analytes first dimension along the axis parallel to the substrate are 100 nm to 200 nm.
216. The system of embodiment 142, wherein said one or more analytes first dimension along the axis parallel to the substrate are less than 200 nm.
217. The system of embodiment 142, wherein said substrate comprises one or more artifacts adjacent to said one or more analytes, wherein said one or more artifacts do not generate a signal.
218. The system of embodiment 217, wherein said one or more artifacts generate a neighboring effect on said one or more analytes.
219. The system of embodiment 218, wherein said neighboring effect comprises immobilizing said one or more analytes.
220. The system of embodiment 219, wherein at least 10% of said one or more artifacts comprise a first dimension along the axis parallel to the substrate of less than the diffraction limit of said optical system configured to image said one or more analytes when said one or more artifacts are deposited adjacent to said one or more analytes, and wherein at least 10% of said one or more artifacts comprise a second dimension along the axis orthogonal to the substrate of less than one-half of the depth-of-focus of said optical system configured to image said one or more analytes when said one or more artifacts are deposited adjacent to said one or more analytes.
221. A method for amplifying a target sequence, the method comprising:
   (a) providing a target sequence;
   (b) providing a three-dimensional solid support; and
   (c) performing rolling circle amplification (RCA) of said target sequence to generate an amplified polynucleotide, wherein said amplified polynucleotide is coupled to said three-dimensional solid support such that said amplified polynucleotide comprises a first dimension along the axis parallel to the substrate and a second dimension along the axis orthogonal to the substrate along an axis orthogonal to the substrate, wherein said first dimension along the axis parallel to the substrate is less than a diffraction limit of an optical system (λ/(2*NA)) configured to image said amplified polynucleotide when said amplified polynucleotide is immobilized on said substrate, and wherein said second dimension along the axis orthogonal to the substrate is less than one-half of a depth-of-focus of said optical system (λ/(2*NA\hat{ }2)).
222. The method of embodiment 221, wherein said three-dimensional support comprises linkers configured to bind to said amplified polynucleotide.
223. The method of embodiment 221, wherein said amplified polynucleotide is bound to one or more three-dimensional solid supports, wherein said one or more three-dimensional solid supports are immobilized on a substrate.
224. The method of embodiment 223, wherein said one or more three-dimensional solid supports are UV treated.
225. The method of embodiment 223, wherein said amplified polynucleotide is bound to a plurality of three-dimensional solid supports of said one or more three-dimensional solid supports.
226. The method of embodiment 223, wherein said amplified polynucleotide is bound to a single three-dimensional solid support of said one or more three-dimensional solid supports.
227. The method of embodiment 222, wherein said linkers are nucleic acid primers.
228. The method of embodiment 221, wherein said amplified polynucleotide comprises repeating regions.
229. The method of embodiment 228, wherein said linkers are configured to bind to said repeating regions of said amplified polynucleotide.
230. The method of embodiment 222, wherein said linkers comprise nucleic acid molecules.
231. The method of embodiment 230, wherein said nucleic acid molecule is DNA or RNA.
232. The method of embodiment 231, wherein said DNA or RNA is double stranded.
233. The method of embodiment 231, wherein said DNA or RNA is single stranded.
234. The method of embodiment 228, wherein said repeating regions comprise a one or more known sequences.
235. The method of embodiment 234, wherein said one or more known sequences comprise a barcode sequence, an adaptor sequence, a primer sequence, or any combination thereof.
236. The method of embodiment 230, wherein a melting point of said linkers is greater than a temperature reached during processing of said amplified polynucleotide.
237. The method of embodiment 223, wherein said three-dimensional support is positively charged.
238. The method of embodiment 223, wherein said three-dimensional support is a nanorod or nanotube.
239. The method of embodiment 223, wherein said three-dimensional support comprises carbon.
240. The method of embodiment 223, wherein said three-dimensional support comprises a precious metal.
241. The method of embodiment 240, wherein said precious metal is gold.
242. The method of embodiment 221, wherein said amplified polynucleotide first dimension along the axis parallel to the substrate is less than 400 nm.
243. The method of embodiment 221, wherein said amplified polynucleotide first dimension along the axis parallel to the substrate is less than 300 nm.
244. The method of embodiment 221, wherein said amplified polynucleotide first dimension along the axis parallel to the substrate is 200 nm to 300 nm.
245. The method of embodiment 221, wherein said amplified polynucleotide first dimension along the axis parallel to the substrate is 100 nm to 200 nm.
246. A method for immobilizing an analyte on a surface, the method comprising:
   (a) providing a plurality of artifacts on said surface;
   (b) passivating said surface;
   (c) removing said plurality of artifacts to generate a plurality of spots, wherein said plurality of spots comprise a diameter of about 50 nanometers (nm) to about 100 nm, and wherein a spot of said plurality of spots is spaced about 180 nm to about 350 nm from one or more adjacent spots of said plurality of spots; and
   (d) displacing an analyte on said spot.
247. The method of embodiment 246, wherein said surface does not comprise a nucleic acid molecule or an antibody covalently or mechanically coupled to said surface.
248. The method of embodiment 246, wherein said artifact comprises a spherical shape.
249. The method of embodiment 248, wherein said spherical shape comprises a diameter of about 200 nm to about 400 nm.
250. The method of embodiment 246, wherein said analyte comprises one or more analytes.
251. The method of embodiment 246, wherein a first dimension along the axis parallel to the substrate of said one or more analytes is less than 400 nm.
252. The method of embodiment 246, wherein a first dimension along the axis parallel to the substrate of said one or more analytes is less than 300 nm.
253. The method of embodiment 246, wherein a first dimension along the axis parallel to the substrate of said one or more analytes is 200 nm to 300 nm.
254. The method of embodiment 246, wherein a first dimension along the axis parallel to the substrate of said one or more analytes is 100 nm to 200 nm.
255. A method for generating a polynucleotide, wherein said polynucleotide comprises one or more copies of a target sequence, the method comprising:
   (a) providing said target sequence;
   (b) ligating one or more predetermined sequences to said target sequence;
   (c) enriching said one or more predetermined sequences or one or more complementary sequences for guanine (G) to generate an enriched sequence;
   (d) amplifying said target sequence and said enriched sequence to generate an amplified polynucleotide; and
   (e) contacting said amplified polynucleotide with a salt.
256. The method of embodiment 255, wherein said enriched sequence comprises one or more homopolymeric regions, wherein said homopolymeric regions comprise a plurality of Gs.
257. The method of embodiment 256, wherein said plurality of Gs is more than three Gs.
258. The method of embodiment 255, wherein said one or more predetermined sequences comprise a barcode sequence, an adaptor sequence, a primer sequence, or any combination thereof.
259. The method of embodiment 258, wherein said adaptor sequence is double stranded.
260. The method of embodiment 258, wherein said adaptor sequence comprises a Y-shape.
261. The method of embodiment 260, wherein a stem of said Y-shaped adapter comprises said homopolymeric region.
262. The method of embodiment 255, wherein said method further comprises one or more cycles of heating and cooling to induce a rigid structure of said amplified polynucleotide.
263. The method of embodiment 255, wherein said amplified polynucleotide comprises a first dimension along the axis parallel to a substrate and a second dimension along the axis orthogonal to said substrate, wherein said first dimension along the axis parallel to the substrate is less than a diffraction limit of an optical system (λ/(2*NA)) configured to image said amplified polynucleotide when said amplified polynucleotide is immobilized on said substrate, and wherein said second dimension along the axis orthogonal to the substrate is less than one-half of a depth-of-focus of said optical system (λ/2*NA\hat{ }2).
264. The method of embodiment 255, wherein said first dimension along the axis parallel to the substrate relative to a plane parallel to said substrate of said amplified polynucleotide is less than 400 nm.
265. The method of embodiment 255, wherein said first dimension along the axis parallel to the substrate relative to a plane parallel to said substrate of said amplified polynucleotide is less than 300 nm.
266. The method of embodiment 255, wherein said first dimension along the axis parallel to the substrate relative to a plane parallel to said substrate of said amplified polynucleotide is 200 nm to 300 nm.
267. The method of embodiment 255, wherein said first dimension along the axis parallel to the substrate relative to a plane parallel to said substrate of said amplified polynucleotide is 100 nm to 200 nm.
268. A method for immobilizing an analyte on a surface, the method comprising:
   (a) providing said surface;
   (b) lowering surface energy of said surface; and
   (c) providing one or more analytes, wherein at least 10% of said one or more analytes comprise a first dimension along the axis parallel to the substrate of less than the diffraction limit of an optical system configured to image said one or more analytes when said one or more analytes are deposited adjacent to said surface, and wherein at least 10% of said one or more analytes comprise a second dimension along the axis orthogonal to the substrate of less than one-half of the depth-of-focus of said optical system configured to image said one or more analytes when said one or more analytes are deposited adjacent to said surface.
269. The method of embodiment 268, wherein said analyte is a nucleic acid concatemer.
270. The method of embodiment 268, wherein said analyte is a protein.
271. The method of embodiment 268, wherein said surface energy of said surface is lowered by contacting said surface with a surface-coating molecule, wherein said surface coating molecule reduces the charge difference between said surface and said analyte.
272. The method of claim 271, wherein said surface-coating molecule comprises one or more hydrophobic moieties.
273. The method of embodiment 271, wherein said surface-coating molecule comprises one or more negatively charged moieties.
274. The method of embodiment 272, wherein said one or more hydrophobic moieties comprise alkane chains comprising at least 6 carbons.
275. The method of embodiment 272, wherein said one or more hydrophobic moieties comprise alkane chains comprising at least 12 carbons.
276. The method of embodiment 272, wherein said one or more hydrophobic moieties comprises a plurality of fluorine groups.
277. The method of claim 271, wherein said surface-coating molecule comprise streptavidin.
278. The method of embodiment 271, wherein said surface-coating molecule are coupled to said surface.
279. The method of embodiment 271, wherein said surface-coating molecule are covalently attached to said surface.
280. The method of embodiment 271, wherein said surface-coating molecule are in solution.
281. The method of embodiment 272, wherein said one or more hydrophobic moieties is part of a block copolymer.
282. The method of embodiment 281, wherein said block copolymer is poloxamer.
283. The method of embodiment 271, wherein said surface-coating molecule are contacted to said surface before providing said one or more analytes.
284. The method of embodiment 271, wherein said surface-coating molecule are contacted to said surface contemporaneously with providing said one or more analytes.
285. The method of embodiment 271, wherein said surface-coating molecule are contacted to said surface after providing said one or more analytes.
286. The method of embodiment 271, wherein said surface-coating molecule are contacted with a linker.
287. The method of embodiment 286, wherein said linker comprises a surface-coating molecule-binding end that binds to said surface-coating molecule.
288. The method of embodiment 271, wherein said surface-coating molecule is streptavidin and said surface-coating molecule-binding end comprises biotin.
289. The method of embodiment 286, wherein said linker comprises a spacer.
290. The method of embodiment 289, wherein said spacer comprises a biopolymer.
291. The method of embodiment 290, wherein said biopolymer comprises a nucleic acid.
292. The method of embodiment 290, wherein said biopolymer comprises polyethylene glycol.
293. The method of embodiment 286, wherein said linker comprises an analyte-binding end.
294. The method of embodiment 293, wherein said analyte-binding end comprises a nucleic acid.
295. The method of embodiment 286, wherein said linker comprises a length of between about 5 and about 10 nm.
296. The method of embodiment 268, wherein said first dimension is less than a diffraction limit of an optical system (λ/(2*NA)) configured to image said analyte, and wherein said second dimension is less than one-half of a depth-of-focus of said optical system (λ/(2*NA\hat{ }2)).
297. The method of embodiment 268, wherein said first dimension is about 300 nm.
298. The method of embodiment 268, wherein said second dimension is about 300 nm.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present disclosure may employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B(1992).

### Example 1: Dense Packing of Molecules

Methods below will describe how to utilize a square ordered array where the pitch ranges between 200 nm and 333 nm. Additional methods will be described that allow even smaller pitches. An imaging system is described in International Application PCT/US2018/020737, filed Mar. 2, 2018 and incorporated herein by reference, which will be used as a reference system which enables sub-diffraction limit imaging. The optical system can include multiple 2,048 by 2,048 pixel cameras operating up to 100 Hz frames per second (fps) with field size 332.8 um by 332.8 um. This system is capable of measuring as little as a single fluor at and above 90 fps. Using this system with 1-10 copies (or 1-10 fluorophores) per molecule at 85 fps achieves the necessary throughput to image a 63 mm×63 mm slide in under 15 minutes. Biochemistry cycles and imaging are continuously and simultaneously performed, either by using two chips or by dividing a single chip into at least 2 regions.

### Example 2: Single-Molecule Sequencing Using Sequencing by Synthesis

Single-molecule sequencing using sequencing-by-synthesis approach was evaluated on the Apton System. To test the methodology, single-stranded DNA templates with 5′ phosphate group were first attached to the chip with a tecarbohydrazide activated silicon surface of the flow cell through EDC (1-Ethyl-3-(3-mplate dimethylaminopropyl)carbodiimide) chemistry. The sequencing primer was then annealed to the target deposited on the surface. The sequencing templates used in our initial studies included synthetic oligonucleotide containing EGFR L858R, EGFR T790M, and BRAF V600E mutations and two cDNA samples reversed transcribed from ERCC 00013 and ERCC 00171 control RNA transcripts. After DNA template immobilization and primer annealing, the flow cell is loaded on the Apton instrument for sequencing reactions, which involves multiple cycles of enzymatic single nucleotide incorporation reaction, imaging to detect fluorescence dye detection, followed by chemical cleavage. Therminator IX DNA Polymerase from NEB was used for single base extension reaction, which is a 9° NTM DNA Polymerase variant with an enhanced ability to incorporate modified dideoxynucleotides. Four dNTPs used in the reaction are labeled with 4 different cleavable fluorescent dyes and blocked at 3′-OH group with a cleavable moiety (dCTP-AF488, dATP-AFCy3, dTTP-TexRed, and dGTP-Cy5 from MyChem). During each sequencing reaction cycle, a single labeled dNTP is incorporated and the reaction is terminated because of the 3′-blocking group on dNTP. After dNTP incorporation, the unincorporated nucleotides are removed from the flow-cell by washing and the incorporated fluorescent dye labeled nucleotide is imaged to identify the base. After the images are captured, the fluorescent dye and blocking moiety are cleaved from the incorporated nucleotide using 100 mM TCEP ((tris(2-carboxyethyl)phosphine), pH9.0), allowing subsequent addition of the next complementary nucleotide in next cycle. This extension, detection and cleavage cycle is then repeated to increase the read length.

The synthetic oligonucleotides used were around 60 nucleotides long. A primer that had a sequence ending one base prior to the mutation in codon 790 was used to enable the extension n reaction. The surface was imaged post incorporation of nucleotides by the DNA polymerase and after the cleavage reaction with TCEP. Molecules were identified with known color incorporation sequences, following that the actual base incorporations are identified by visual inspections which is labor-intensive.

Dye labeled nucleotides were used to sequence cDNA generated from RNA templates. RNA used was generated by T7 transcription from cloned ERCC control plasmids. The data exhibits the ability of the system to detect 10 cycles of base incorporation. The sequence observed were correct. Yellow arrows indicate the cleavage cycles.

Specifically, cDNA templates corresponding to transcripts generated from the ERCC (External RNA Controls Consortium) control plasmids by T7 transcription were sequenced. The cDNA molecule generated were >350 nucleotides long. The surface was imaged post incorporation of nucleotides by the DNA polymerase and after the cleavage reaction with TCEP. Data indicated ability to manually detect 10 cycles of nucleotide incorporation by manual viewing of images.

### Example 3: Relative Location Determination for Analyte Variants

In some embodiments a single molecules deposited on a substrate is bound by a probe comprising a fluorophore. The molecules are anti-ERK antibodies bound to ERK protein from cell lysate which has been covalently attached to the solid support. The antibodies are labeled with 3-5 fluorophores per molecule. Similar images are attainable with single fluor nucleic acid targets, e.g., during sequencing by synthesis.

To improve accuracy of detection, the molecules undergo successive cycles of probe binding and stripping, in this case 30 cycles. In each round, the image is processed to determine the location of the molecules. The images are background subtracted, oversampled by 2×, after which peaks are identified. Multiple layers of cycles are overlaid on a 20 nm grid. The location variance is the standard deviation, or the radius divided by the square root of the number of measurements.

### Example 4: Densely-Packed Sequencing Substrates and Single-Sided Density

### Single-Stranded Circle Formation:

To prepare a library of concatemers comprising target sequences to distribute on the surface of a substrate in a randomly distributed close-packed layer, a sample comprising target sequences was amplified, purified, ligated to form circularized DNA, and quantified.

### Amplification of Targets

An Illumina MiSeq library was purchased from SegMatic (Fremont, CA) made with the standard protocol using E. coli DNA purchased from Affymetrix (Santa Clara, CA—PN 14380)

The library was amplified by PCR amplification. Each PCR reaction included the following components listed in Table 1:

TABLE 1

| | One 50 ul |
|---|---|
| | Reaction (uL) |
| 10X Pfx Amplification buffer | 10 |
| 10 mM dNTP (Invitrogen) | 1.5 |
| 50 mM MgSO4 (stored at 4° C.) | 1 |
| Primer mix (1004) | 1.5 |
| Template DNA | 1-5 |
| Platinum Pfx DNA Polymerase | 0.4 |
| (Invitrogen - ThermoFisher) | |
| Pfx Enhancer (Invitrogen - | 2.5 |
| ThermoFisher) | |
| Water | Fill with water |
| | to 50 ul_, |

The primer mix is a 50:50 mix of P5-Phosphate (/5Phos/AAT GAT ACG GCG ACC ACC GA) and P7 (CAA GCA GAA GAC GGC ATA CGA GAT) primers at 10 uM:

The PCR amplification was performed under the following conditions: 5 mM at 94° C. followed by 35 cycles of: 94° C., 15 sec; 55° C., 30 sec; and 68° C., 30 sec. An aliquot of the amplification product was run on a 2% gel to verify the library molecule size (300-500 base pairs in this instance). The PCR amplification product was then purified using a PureLink® Spin Column (Thermofisher) according to the manufacturer's protocol.

### Circularization of Target DNA

The purified PCR amplification products were then subject to single strand circularization by ligation in the reaction mix described in Table 2:

TABLE 2

| | Single |
|---|---|
| | reaction (uL) |
| 10 X HiFi Taq DNA Ligase Buffer | 5 |
| DNA template (104) | 10 |
| Bridging oligonucleotide (100 uM) | 1 |
| HiFi Taq DNA Ligase (New England | 1 |
| Biolabs, Ipswich MA) | |
| H2O | 33 |
| Total vol (at) | 50 |

The bridging oligonucleotide sequence was TCG GTG GTC GCC GTA TCA TTC AAG CAG AAG ACG GCA TAC GAG AT.

The ligation was performed under the following conditions: 30 sec at 95° C. followed by 40 cycles of: 95° C., 15 sec; 55° C., 2 min; and 62° C., 3 min.

After ligation, 1 μL each of Exonuclease I and Exonuclease III (New England Biolabs) were added and the reaction is incubated for an additional 45 min at 37° C. and 30 min at 85° C. The resulting material was purified using a Zymo-Spin™ Column (Oligo Clean & Concentrator™ kit Zymo Research, Irvine, CA) using the manufacturer's protocol. After purification, the concentration was measured using a Qubit 2.0 fluorometer (ThermoFisher) and Quant-iT OliGreen® (ThermoFisher) with custom calibration samples using an oligonucleotide of known concentration.

Concatamer Formation from Circularized DNA

Concatemers from circularized DNA comprising the target sequence were formed in a reaction mix described in Table 3:

TABLE 3

| | | | | Additional |
|---|---|---|---|---|
| | volume | | buffer | components |
| circular template | 10 | μL | water | |
| primer solution | 5 | μL | 3X reaction | |
| | | | buffer | |
| Enzyme mix | 5 | μL | lx reaction | 2 U/ul Phi29 DNA |
| | | | buffer | polymerase 2 mM in |
| | | | | each dNTP 0.004 U/uL |
| | | | | iPPase |
| | | | | (all from New England |
| | | | | Biolabs, Ipswich, MA) |
| Reaction | 5 | μL | 0.25M EDTA, | |
| inactivation | | | pH 8.0 | |
| buffer | | | (Sigma-Aldrich, | |
| | | | St. Louis, MO) | |

The primer solution was a 750 nM suspension of the primer (ATC TCG TAT GCC GTC TTC TGC TTG) in 3× reaction buffer. The 10× reaction buffer was: 500 mM Tris-HCl, 100 mM (NH4)2SO4, 40 mM DTT, 100 mM MgCl2, pH 7.5 @ 25° C.

The circular template+primer mix was incubated for 10 mM at 90° C., and then 30 min at 30° C. A pre-warmed enzyme mix was then added as in Table 3 for 90 mM. The reaction was stopped with the addition of reaction inactivation buffer and stored at 4° C.

Concatemer libraries were then layered on a substrate to form a densely-packed, randomly distributed layer bound to the surface of a substrate, followed by sequencing the bound concatemers via imaging and image processing, and analysis of the data.

One microliter of the sequencing substrate was mixed with 19 ul of citrate phosphate buffer, and 10 ul was loaded onto a custom biochip and incubated overnight. The chip was then washed 2× with citrate phosphate buffer, 2× with potassium phosphate buffer and 2× with NA wash 3 buffer.

Fluorescent probe was bound to the concatemer layer bound to the surface of the chip to determine identity.

### Example 5: Sequencing E. Coli Reads

### Imaging/Sequencing

Sequencing by synthesis was performed using standard sequencing chemistries. The chip comprising the densely packed concatemer layer was loaded into the AptonBio Sequencer and washed 6×5 mM at 60° C. with Washl (20 mM Tris-HCl, 10 mM (NH4)2 SO4, 10 mM KCl, 2 mM MgSo4, 0.1% 100, pH 8.8 @ 25° C., 50 mM NaCl). The sequencing oligo (ATC TCG TAT GCC GTC TTC TGC TTG) was diluted to 100 nM in hybridization buffer and incubated 1×1 mM followed by 2×10 mM at 60° C. with Washl washes between hybridization operations. Then thirty-two cycles of the following 8 operations were performed:
1—Cleavage: 225 sec at 60° C. with buffer in Table 4

TABLE 4

| | |
|---|---|
| | Concentration |
| Amount | (Working) |

| | | | | |
|---|---|---|---|---|
| TCEP [add vendor] | 31.53 | mg | 100 | mM |
| lON NaOH | 40 | uL | | |
| 5M Nacl | 11 | uL | 50 | mM |

| 1M Tris-HCL | 11 | −1M |
|---|---|---|
| Total Volume | 1100 | |

2—Wash: 240 sec at 30° C. in Phosphate buffer pH 8.
3—Imaging: Wash2 (20 mM Tris-HCl, 5 mM Ascorbic Acid (pH 8.8)
4—Wash: Washl at 60° C.
5—Extension: 450 sec at 60° C. with buffer in Table 5

TABLE 5

| | | | | | Vol/ |
|---|---|---|---|---|---|
| | Concentration | | Concentration | | reaction |
| | (Stock) | | (Working) | | (μL) |
| ThermoPol Reaction | 10 x | | 1 x | | 5 |
| Buffer (NEB) | | | | | |
| dATP labeled | 5 | μM | 0.1 | uM | 1 |
| reversible | | | | | |
| terminator | | | | | |
| (MyChem, LLC, | | | | | |
| San Diego) | | | | | |
| dGTP labeled | 5 | uM | 0.1 | uM | 1 |
| reversible | | | | | |
| terminator | | | | | |
| (MyChem, LLC, | | | | | |
| San Diego) | | | | | |
| dTTP labeled | 5 | μM | 0.1 | uM | 1 |
| reversible | | | | | |
| terminator | | | | | |
| (MyChem, LLC, | | | | | |
| San Diego) | | | | | |
| dCTP labeled | 5 | uM | 0.1 | uM | 1 |
| reversible | | | | | |
| terminator | | | | | |
| (MyChem, LLC, | | | | | |
| San Diego) | | | | | |
| NaCl | 5M | | 0.05M | | 0.5 |
| Therminator | 10 | U/μ.L | 0.05 | U | 1.25 |
| X (New England | | | | | |
| Biolabs Ipswich, | | | | | |
| MA) | | | | | |
| Non-labeled | 10 | uM | 0 to 1 | uM | 0-5 |
| dNTP Mix | | | | | |
| (MyChem, LLC, | | | | | |
| San Diego) | | | | | |
| Water | | | | | 38.75-34.24 |
| Total | | | | | 50 |

6—Wash: Washl at 30° C.
7—Wash: 2 min at 30° C. in Phosphate buffer pH8.
8—Imaging: Wash 2.

### Example 6: Compactability of DNA Concatamers

High density, multi-color super-resolution fluorescence imaging of DNA concatemers self-assembled on a substrate promises significant improvements in density and cost over traditional sequencing methods. Using DNA as a mechanical material, DNA circularly amplified concatemers (CATs) self-assemble on a substrate avoiding the use of high-cost lithographic techniques. For the case of a super-resolution optical system with resolution below lambda/(2 NA), it is desirable to match the size of DNA with the optical system while simultaneously containing as much DNA as possible that is still accessible for efficient chemistry.

To achieve high density sequencing CATs must load well within a diffraction limited spot with a max radius of about 200 nm. The DNA must remain in that region throughout the experiment and accessible to polymerases for SBS and hair growth. Physically the amount of DNA produced through RCA, which can range between 12 kb and 150 kb for a 5 and 60 min CAT, can fit in a cube with a diameter of 20-50 nm, respectively (FIG. 4, solid line). However, only 1% of maximum compactification level is sufficient (FIG. 4, dotted line), due to diffraction limits and enzyme accessibility.

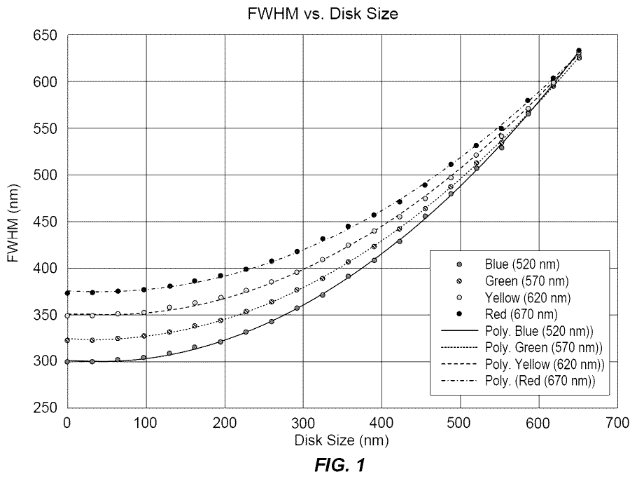

In one embodiment, lambda is 600 nm and NA is 1.0 giving a desired concatemer size of 200 nm-250 nm. This is demonstrated in FIG. 1 where an assumed disk of DNA is convolved with the point-spread function (PSF) of the optical system. A disk of diameter 250 nm has a negligible effect on the measured FWHM, increasing it by ˜10%.

FIG. 2 shows measured full-width half maximum widths (FWHM) for CATs of 5 min, 10 min, 15 min & 60 min CATs of approximately 12 kB, 24 kB, 36 kB and 144 kB of DNA nucleotide length. The thickness of DNA on the surface is estimated to be between 2 nm-8 nm for FWHM with diameters of 380 nm-580 nm respectively. For this system, the depth of focus (DOF) is =lambda/(2 NA\hat{ }2)=300 nm. A CAT thickness of 100 nm would have a negligible effect on the PSF of the system while allowing for >10× more DNA in the CAT at the same nucleotide volumetric density.

### Example 7: Nanoarray Passivation of a Surface

To achieve high density, uniform, single-molecules arrays on surface for sequencing, DNA nanoarrays are built using self-assembled colloidal nanoparticles(microspheres). For the sphere size 200 nm, it may achieve the density (number of molecule per micrometer square) up to 40 (molecules/μm²).

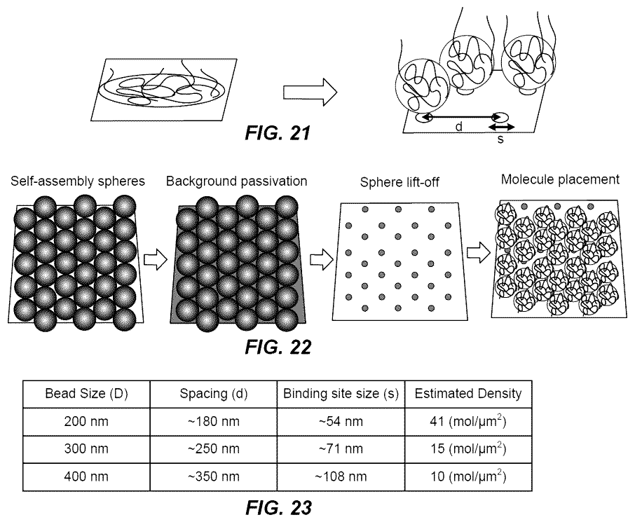

FIG. 21 depicts the difference between molecules on a surface without nanoarray passivation (left) and with nanoarray passivation (right). Molecules on a surface with nanoarray passivation are placed closer together.

The process of nanoarray passivation is depicted in FIG. 23. Nanospheres are placed on the surface. The surface is passivated through methods described herein. The nanospheres are removed, leaving an array of selectively passive surface. The DNA molecules are then added to the array and cluster densely. The estimated density for different size nanospheres is listed in FIG. 23.

### Example 8: Binding of Concatemers to a Streptavidin-Functionalized Surface

A method of binding compact CATs to a streptavidin-functionalized surface is depicted in FIG. 26. A surface is functionalized with streptavidin. The streptavidin functionalized surface has a lowered surface energy. DNA concatamers can no longer bind to this surface, so a linker is required to attach. A linker comprising a biotin end that binds to the streptavidin surface, a spacer of DNA, and a DNA primer end that attaches to the CAT is used. Alternatively, a linker comprising a biotin end that binds to the streptavidin surface, a biopolymer spacer (e.g. PEG), and a DNA primer end that attaches to the CAT is used. The CATs are held off the surface and have a diameter of about 300 nm. This results in compactification of the CATs.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-119. (canceled)

120. An imaging system comprising (i) an analyte disposed on a substrate, and (ii) an optical imaging module configured to image the analyte;
wherein the analyte has a first dimension along an axis orthogonal to the substrate;
wherein the first dimension is less than about one-half of a depth-of-focus of the optical imaging module (λ/(2*NA\hat{ }2)); and
wherein a support is coupled to the analyte and configured to maintain the first dimension.

121. The imaging system of claim 120, wherein the substrate comprises a selectively passive surface.

122. The imaging system of claim 120, wherein the analyte comprises a second dimension, wherein the second dimension is along an axis parallel to the substrate, and wherein the second dimension is less than a diffraction limit of the optical imaging module (λ/2*NA).

123. The imaging system of claim 120, wherein the support is a spherical or circular structure.

124. The imaging system of claim 120, wherein the support is a nanotube or a nanoparticle, wherein the nanotube or the nanoparticle comprises functionalized primers.

125. The imaging system of claim 120, wherein the support is an origami structure.

126. The imaging system of claim 120, wherein the support comprises primers, wherein the primers are configured to bind to the analyte.

127. The imaging system of claim 120, wherein the support comprises a metal.

128. The imaging system of claim 120, wherein the support comprises a nucleic acid molecule.

129. The imaging system of claim 120, wherein the support comprises a nanorod.

130. The imaging system of claim 120, wherein the analyte comprises repeating regions.

131. The imaging system of claim 130, wherein the support comprises one or more linkers, wherein the one or more linkers are configured to bind to the repeating regions.

132. The imaging system of claim 131, wherein the one or more linkers comprise nucleic acid molecules, wherein the nucleic acid molecules comprise a barcode sequence, an adapter sequence, a primer sequence, or any combination thereof.

133. The imaging system of claim 120, wherein the analyte is immobilized on the substrate.

134. The imaging system of claim 120, wherein the substrate comprises one or more artifacts adjacent to the analyte, wherein the artifacts generate a neighboring effect on the analyte.

135. The imaging system of claim 120, wherein the analyte comprises a scaffold.

136. The imaging system of claim 120, wherein the analyte is a concatemer.

137. The imaging system of claim 120, wherein the substrate comprises anchor moieties.

138. A system for densely packing analytes, the system comprising: a plurality of analytes disposed on a surface;
wherein the analytes are separated from one another by a center-to-center distance of less than or equal to 500 nanometers; and
wherein a support is configured to condense an analyte of the plurality of analytes.

139. A method for processing one or more analytes, the method comprising:
a) depositing the one or more analytes adjacent to a substrate, wherein at least 10% of the one or more analytes comprise a first dimension along an axis parallel to the substrate of less than a diffraction limit of an optical system configured to image the one or more analytes when the one or more analytes are deposited adjacent to the substrate, and wherein at least 10% of the one or more analytes comprise a second dimension along an axis orthogonal the substrate of less than one-half of the depth-of-focus of the optical system configured to image the one or more analytes when the one or more analytes are deposited adjacent to the substrate;
b) contacting the one or more analytes with a plurality of probes over a plurality of cycles, wherein the plurality of probes generate a plurality of signals;
c) obtaining the plurality of optical signals from the plurality of probes over the plurality of cycles of the plurality of probes binding to the one or more analytes deposited adjacent to the substrate; and
d) processing at least one optical signal of the plurality of optical signals to identify the one or more analytes of the plurality of analytes.
